(19) United States
(12) Patent Application Publication
(10) Pub. No.: US 2026/0347883 A1
(43) Pub. Date: Nov. 5, 2026

(54) Strategies For Knock-Ins At B2M Safe Harbor Sites

(71) Applicant: EmendoBio Inc., Wimington, DE (US)

(72) Inventors: Rafi EMMANUEL, Ramla (IL); Michal GOLAN MASHIACH, Ness-Ziona (IL)

(21) Appl. No.: 19/107,769

(22) PCT Filed: Aug. 29, 2023
(86) PCT No.: PCT/US2023/073068

§ 371 Date: Feb. 28, 2025

Related U.S. Application Data
(60) Provisional application No. 63/373,990, filed on Aug. 30, 2022.
(60) Provisional application No. 63/477,888, filed on Dec. 30, 2022.

(51) Int. Cl. C12N 15/90; C12N 9/22; C12N 15/11

(52) U.S. Cl. CPC C12N 15/907; C12N 9/226; C12N 15/11; C12N 2310/20

(57) ABSTRACT

RNA molecules comprising a guide sequence portion having 17-50 contiguous nucleotides in the sequence set forth in any one of SEQ ID NOs: 1-28998 and compositions, methods, and uses thereof.

This application claims the benefit of U.S. Provisional Application Nos. 63/477,888 filed Dec. 30, 2022, and 63/373,990, filed Aug. 30, 2022, the content of each of which is hereby incorporated by reference.

Throughout this application, various publications are referenced, including referenced in parenthesis. The disclosures of all publications mentioned in this application in their entireties are hereby incorporated by reference into this application in order to provide additional description of the art to which this invention pertains and of the features in the art which can be employed with this invention.

### REFERENCE TO SEQUENCE LISTING

This application incorporates-by-reference nucleotide sequences which are present in the file named “230829_91780-A-PCT_Sequence_Listing_AWG.xml”, which is 25,695 kilobytes in size, and which was created on Aug. 16, 2023 in the IBM-PC machine format, having an operating system compatibility with MS-Windows, which is contained in the XML file filed Aug. 29, 2023 as part of this application.

### BACKGROUND OF INVENTION

A Beta-2 microglobulin (B2M) safe harbor site may be targeted to introduce a desired sequence to the site without causing deleterious disruptions to the B2M gene or affecting its expression. Such targeting strategies may be utilized to enable B2M promoter mediated expression of the introduced sequence.

### SUMMARY OF THE INVENTION

The present disclosure also provides a method for modifying in a cell at least one allele of the Beta-2 microglobulin (B2M) gene, the method comprising
introducing to the cell a composition comprising:
   at least one CRISPR nuclease, or a polynucleotide molecule encoding the CRISPR nuclease; and
an RNA molecule comprising a guide sequence portion having 17-50 nucleotides or a nucleotide sequence encoding the same,
wherein a complex of the CRISPR nuclease and the RNA molecule affects a double strand break in at least one allele of the B2M gene.

In some embodiments, the composition also comprises a donor molecule, wherein a sequence of nucleotides from the donor molecule is inserted or copied at or near the double strand break site. Similarly, in some embodiments, the composition further comprises a donor molecule containing a sequence of nucleotides that is introduced at the double strand break site such that the expression of the introduced sequence is mediated by the promoter of the B2M gene. Accordingly, also disclosed herein are strategies to enable the expression of a gene, or portion thereof, under control of the B2M promoter. In some embodiments, the expression of a gene, or portion thereof, under control of the B2M promoter is enabled without knocking-out B2M gene expression.

According to embodiments of the present invention, there is provided a first RNA molecule comprising a guide sequence portion having 17-50 contiguous nucleotides containing nucleotides in the sequence set forth in any one of SEQ ID NOs: 1-28998. In some embodiments, the composition further comprises a CRISPR nuclease. In some embodiments, the composition further comprises a donor molecule.

According to some aspects of the present invention, there is provided a cell modified to express and secrete a protein of interest within a desired body tissue. Such a cell can be modified by insertion of a sequence of a gene of interest under the control of a promoter of a selected gene which contains a safe harbor site (e.g., B2M). Non-limiting examples of methods for modifying a cell to express a gene of interest include knock-in by utilizing a CRISPR nuclease system that generates a double-strand break and a donor molecule which encodes a sequence of the gene of interest. In some embodiments, the donor molecule is a ssODN, dsDNA, plasmid, AAV, lentivirus, or transposon. In addition, knock-in can be mediated by a composition comprising i) a fusion protein comprising a nickase and a reverse transcriptase and ii) a RNA donor molecule.

According to embodiments of the present invention, there is provided a cell modified by any one of the methods described herein. In some embodiments, the cell is a stem cell. In some embodiments, the cell is a monocyte. In some embodiments, the cell is a macrophage. In some embodiments, the cell is an iPS-derived monocyte or macrophage. In some embodiments, the cell is a hematopoietic stem cell (HSC), a hematopoietic stem and progenitor cell (HSPC), a myeloid precursor cell, a myeloblast, a lymphoblast, an erythroid precursor cell, a platelet cell, a natural killer (NK) cell, a B-lymphocyte, a T-lymphocyte, an eosinophil, a neutrophil, or a basophil. In some embodiments, the cell is an iPS-derived cell.

In some embodiments, the delivering of any one of the compositions described herein to the cell is performed in vitro, ex vivo, or in vivo. In some embodiments, the method is performed ex vivo and the cell is provided/explanted from an individual patient. In some embodiments, the method further comprises the step of introducing the resulting cell, with a modified or edited B2M allele, into the individual patient (e.g. autologous transplantation).

According to some embodiments of the present invention, there is provided use of a composition comprising an RNA molecule comprising a guide sequence portion having 17-50 contiguous nucleotides containing nucleotides in the sequence set forth in any one of SEQ ID NOs: 1-28998 and a CRISPR nuclease for modifying or editing a B2M allele in a cell, comprising delivering to the cell the composition comprising an RNA molecule comprising a guide sequence portion having 17-50 contiguous nucleotides containing nucleotides in the sequence set forth in any one of SEQ ID NOs: 1-28998 and a CRISPR nuclease. In some embodiments, the composition further comprises a donor molecule.

According to embodiments of the present invention, there is provided a medicament comprising an RNA molecule comprising a guide sequence portion having 17-50 contiguous nucleotides containing nucleotides in the sequence set forth in any one of SEQ ID NOs: 1-28998 and a CRISPR nuclease for use in modifying or editing a B2M allele in a cell, wherein the medicament is administered by delivering to the cell the composition comprising an RNA molecule comprising a guide sequence portion having 17-50 contiguous nucleotides containing nucleotides in the sequence set forth in any one of SEQ ID NOs: 1-28998 and a CRISPR nuclease. In some embodiments, the medicament further comprises a donor molecule.

According to some embodiments of the present invention, there is provided a kit for modifying or editing B2M allele in a cell, comprising an RNA molecule comprising a guide sequence portion having 17-50 contiguous nucleotides containing nucleotides in the sequence set forth in any one of SEQ ID NOs: 1-28998, a CRISPR nuclease, and/or a tracrRNA molecule; and instructions for delivering the RNA molecule; CRISPR nuclease, and/or the tracrRNA to the cell. In some embodiments, the kit further comprises a donor molecule.

### BRIEF DESCRIPTION OF THE DRAWINGS

FIG. 1A: Screening guides for optimal editing activity in Intron 1 of B2M in HeLa cells using OMNI-50 nuclease. Cells were harvested 72 hours post-DNA transfection, genomic DNA was extracted, and the region of the mutation was analyzed by next generation sequencing (NGS). FIG. 1B: Examination of editing activity of selected guides in HSCs using OMNI-50 or OMNI-50 V6172 CRISPR nucleases identifies optimal composition for editing in Intron 1 of B2M. HSCs were electroporated with RNP (CRISPR nuclease and RNA guide), 72 hours post-electroporation the genomic DNA was extracted, and the region of the mutation was analyzed by NGS. Graphs represent % editing±standard deviation (SD) of two independent repeats.

FIG. 2: Insertion of GFP by HDR into a B2M endogenous locus for generating a bicistronic transcript. Pre-editing shows the native form of a B2M locus encodes for B2M protein including its signal peptide (SP) in its N-terminus. The SP portion of B2M is encoded by Exon 1. A donor sequence carrying GFP-2A-SP flanked by left and right homology arms (LHA and RHA, respectively), and splice acceptor and splice donor sites, is also shown. Successful integration results in a new intermediate exon and a new, chimeric, polypeptide of SP-GFP-2A-SP-B2M. The chimeric polypeptide is self-cleaved into two distinct proteins with an SP.

FIGS. 3A-3D: Integration of GFP into a B2M locus in HSCs enables GFP expression and secretion without altering B2M expression. FIG. 3A: FACS analysis of HSCs treated with donor virus (GFP-2A) and RNP results in GFP-expressing cells. GFP signal was not detected in HSCs treated with donor virus alone or with an irrelevant RNP. FIG. 3B: Examination of total B2M transcript levels normalized to GAPDH reveals no change in B2M transcript levels following HDR integration. Data shown is two biological repeats and three technical repeats (nested analysis). FIG. 3C: Examination of B2M protein expression using FACS confirms most edited cells retain B2M protein expression. B2M expression in GFP+, edited cells following HDR (top), compared to all live cells in the untreated and ISO-staining control groups. Dashed line represents expression cut-off as determined by the ISO-staining control. FIG. 3D: GFP secretion assayed by ELISA detects GFP (GFP concentration (pg/ml) normalized to 10⁶ cells) in HSC media following HDR into a B2M locus. Data shown is two biological repeats and two technical repeats (nested analysis). Graphs show mean±SD.

FIGS. 4A-4C: Integration of GFP into a B2M locus in HSCs, followed by differentiation into macrophages, enables the generation of GFP-secreting macrophages. FIG. 4A: FACS analysis of macrophages to verify effectiveness of a differentiation protocol and to validate GFP expression. FIG. 4B: Examination of B2M protein expression using FACS confirms most edited cells retain B2M protein expression. B2M expression in GFP+, edited cells following HDR (top), compared to all live cells in the untreated and ISO staining control groups. Cells were categorized to WT-level expressing cells (based on untreated population), reduced expression, and no expression (based on ISO-staining control). FIG. 4C: GFP secretion as measured by ELISA detects GFP in macrophage media following HDR into a B2M locus in HSCs. Data shown is two biological repeats and two technical repeats (nested analysis). Graph shows mean±SD.

FIGS. 5A-5B: Integration of GFP into a B2M locus in iPSCs enables GFP expression and secretion. FIG. 5A: FACS of iPSCs treated with donor virus (GFP-2A)+RNP results in GFP expression. GFP was not observed in cells treated with donor virus when combined with irrelevant RNP. FIG. 5B: GFP secretion as measured by ELISA detects GFP in cell media following HDR into a B2M locus. Data shown is two biological repeats and two technical repeats (nested analysis). Graphs show mean±SD.

### DETAILED DESCRIPTION

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

It should be understood that the terms “a” and “an” as used above and elsewhere herein refer to “one or more” of the enumerated components. It will be clear to one of ordinary skill in the art that the use of the singular includes the plural unless specifically stated otherwise. Therefore, the terms “a,” “an” and “at least one” are used interchangeably in this application.

For purposes of better understanding the present teachings and in no way limiting the scope of the teachings, unless otherwise indicated, all numbers expressing quantities, percentages or proportions, and other numerical values used in the specification and claims, are to be understood as being modified in all instances by the term “about.” Accordingly, unless indicated to the contrary, the numerical parameters set forth in the following specification and attached claims are approximations that may vary depending upon the desired properties sought to be obtained. At the very least, each numerical parameter should at least be construed in light of the number of reported significant digits and by applying ordinary rounding techniques.

Unless otherwise stated, adjectives such as “substantially” and “about” modifying a condition or relationship characteristic of a feature or features of an embodiment of the invention, are understood to mean that the condition or characteristic is defined to within tolerances that are acceptable for operation of the embodiment for an application for which it is intended. Unless otherwise indicated, the word “or” in the specification and claims is considered to be the inclusive “or” rather than the exclusive or, and indicates at least one of, or any combination of items it conjoins.

In the description and claims of the present application, each of the verbs, “comprise,” “include” and “have” and conjugates thereof, are used to indicate that the object or objects of the verb are not necessarily a complete listing of components, elements or parts of the subject or subjects of the verb. Other terms as used herein are meant to be defined by their well-known meanings in the art.

The term “homology-directed repair” or “HDR” refers to a mechanism for repairing DNA damage in cells, for example, during repair of double-stranded and single-stranded breaks in DNA. HDR requires nucleotide sequence homology and uses a “nucleic acid template” (nucleic acid template or donor template used interchangeably herein) to repair the sequence where the double-stranded or single break occurred (e.g., DNA target sequence). This results in the transfer of genetic information from, for example, the nucleic acid template to the DNA target sequence. HDR may result in alteration of the DNA target sequence (e.g., insertion, deletion, mutation) if the nucleic acid template sequence differs from the DNA target sequence and part or all of the nucleic acid template polynucleotide or oligonucleotide is incorporated into the DNA target sequence. In some embodiments, an entire nucleic acid template polynucleotide, a portion of the nucleic acid template polynucleotide, or a copy of the nucleic acid template is integrated at the site of the DNA target sequence.

The terms “nucleic acid template” and “donor”, refer to a nucleotide sequence that is inserted or copied into a genome. The nucleic acid template comprises a nucleotide sequence, e.g., of one or more nucleotides, that will be added to or will template a change in the target nucleic acid or may be used to modify the target sequence. A nucleic acid template sequence may be of any length, for example between 2 and 10,000 nucleotides in length. A nucleic acid template may be a single stranded nucleic acid, a double stranded nucleic acid. In some embodiments, the nucleic acid template comprises a nucleotide sequence, e.g., of one or more nucleotides, that corresponds to wild type sequence of the target nucleic acid, e.g., of the target position. In some embodiments, the nucleic acid template comprises a nucleotide sequence, e.g., of one or more ribonucleotides, that corresponds to wild type sequence of the target nucleic acid, e.g., of the target position. In some embodiments, the nucleic acid template comprises modified nucleotides.

Insertion of an exogenous sequence (also called a “donor sequence,” donor template,” “donor molecule” or “donor”) can also be carried out. For example, a donor sequence can contain a non-homologous sequence flanked by two regions of homology to allow for efficient homology directed repair (HDR) at the location of interest. Additionally, donor sequences can comprise a vector molecule containing sequences that are not homologous to the region of interest in cellular chromatin. A donor molecule can contain several, discontinuous regions of homology to cellular chromatin. For example, for targeted insertion of sequences not normally present in a region of interest, said sequences can be present in a donor nucleic acid molecule and flanked by regions of homology to sequence in the region of interest. A donor molecule may be any length, for example ranging from several bases e.g. 10-20 bases to multiple kilobases in length.

The donor polynucleotide can be DNA or RNA, single-stranded and/or double-stranded and can be introduced into a cell in linear or circular form. See, e.g., U.S. Publication Nos. 2010/0047805; 2011/0281361; 2011/0207221; and 2019/0330620. See also Anzalone et al. (2019). If introduced in linear form, the ends of the donor sequence can be protected (e.g., from exonucleolytic degradation) by methods known to those of skill in the art. For example, one or more dideoxynucleotide residues are added to the 3′ terminus of a linear molecule and/or self-complementary oligonucleotides are ligated to one or both ends. See, for example, Chang et al. (1987) and Nehls et al. (1996). Additional methods for protecting exogenous polynucleotides from degradation include, but are not limited to, addition of terminal amino group(s) and the use of modified internucleotide linkages such as, for example, phosphorothioates, phosphoramidates, and O-methyl ribose or deoxyribose residues.

A donor sequence may be an oligonucleotide and be used for targeted alteration of an endogenous sequence. The oligonucleotide may be introduced to the cell on a vector, may be electroporated into the cell, or may be introduced via other methods known in the art. Donor polynucleotides can be introduced as naked nucleic acid, as nucleic acid complexed with an agent such as a liposome or poloxamer, or can be delivered by viruses (e.g., adenovirus, AAV, herpesvirus, retrovirus, lentivirus and integrase defective lentivirus (IDLV)).

As used herein, the term “modified cells” refers to cells in which a double strand break is affected by a complex of an RNA molecule and the CRISPR nuclease as a result of hybridization with the target sequence, i.e. on-target hybridization. The term “modified cells” may further encompass cells in which an edit or modification, including the introduction of an exogenous sequence, was affected following the double strand break.

This invention provides a modified cell or cells obtained by use of any of the methods described herein. In an embodiment these modified cell or cells are capable of giving rise to progeny cells. In an embodiment these modified cell or cells are capable of giving rise to progeny cells after engraftment. As a non-limiting example, the modified cells may be hematopoietic stem cells (HSCs), or any cell suitable for an allogenic cell transplant or autologous cell transplant. As a non-limiting example, the modified cells may be stem cells, monocytes, macrophages, or iPS-derived monocytes or macrophages.

This invention also provides a composition comprising these modified cells and a pharmaceutically acceptable carrier. Also provided is an in vitro or ex vivo method of preparing this, comprising mixing the cells with the pharmaceutically acceptable carrier.

As used herein, the term “targeting sequence” or “targeting molecule” refers a nucleotide sequence or molecule comprising a nucleotide sequence that is capable of hybridizing to a specific target sequence, e.g., the targeting sequence has a nucleotide sequence which is at least partially complementary to the sequence being targeted along the length of the targeting sequence. The targeting sequence or targeting molecule may be part of an RNA molecule that can form a complex with a CRISPR nuclease, either alone or in combination with other RNA molecules, with the targeting sequence serving as the targeting portion of the CRISPR complex. When the molecule having the targeting sequence is present contemporaneously with the CRISPR molecule, the RNA molecule, alone or in combination with an additional one or more RNA molecules (e.g. a tracrRNA molecule), is capable of targeting the CRISPR nuclease to the specific target sequence. As non-limiting example, a guide sequence portion of a CRISPR RNA molecule or single-guide RNA molecule may serve as a targeting molecule. Each possibility represents a separate embodiment. A targeting sequence can be custom designed to target any desired sequence.

The term “targets” as used herein, refers to preferentially hybridizing a targeting sequence of a targeting molecule to a nucleic acid having a targeted nucleotide sequence. It is understood that the term “targets” encompasses variable hybridization efficiencies, such that there is preferential targeting of the nucleic acid having the targeted nucleotide sequence, but unintentional off-target hybridization in addition to on-target hybridization might also occur. It is understood that where an RNA molecule targets a sequence, a complex of the RNA molecule and a CRISPR nuclease molecule targets the sequence for nuclease activity.

The “guide sequence portion” of an RNA molecule refers to a nucleotide sequence that is capable of hybridizing to a specific target DNA sequence, e.g., the guide sequence portion has a nucleotide sequence which is partially or fully complementary to the DNA sequence being targeted along the length of the guide sequence portion. In some embodiments, the guide sequence portion is 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 nucleotides in length, or approximately 17-50, 17-49, 17-48, 17-47, 17-46, 17-45, 17-44, 17-43, 17-42, 17-41, 17-40, 17-39, 17-38, 17-37, 17-36, 17-35, 17-34, 17-33, 17-31, 17-30, 17-29, 17-28, 17-27, 17-26, 17-25, 17-24, 17-22, 17-21, 18-25, 18-24, 18-23, 18-22, 18-21, 19-25, 19-24, 19-23, 19-22, 19-21, 19-20, 20-22, 18-20, 20-21, 21-22, or 17-20 nucleotides in length. Preferably, the entire length of the guide sequence portion is fully complementary to the DNA sequence being targeted along the length of the guide sequence portion. The guide sequence portion may be part of an RNA molecule that can form a complex with a CRISPR nuclease with the guide sequence portion serving as the DNA targeting portion of the CRISPR complex. When the RNA molecule having the guide sequence portion is present contemporaneously with the CRISPR molecule, alone or in combination with an additional one or more RNA molecules (e.g. a tracrRNA molecule), the RNA molecule is capable of targeting the CRISPR nuclease to the specific target DNA sequence. Accordingly, a CRISPR complex can be formed by direct binding of the RNA molecule having the guide sequence portion to a CRISPR nuclease or by binding of the RNA molecule having the guide sequence portion and an additional one or more RNA molecules to the CRISPR nuclease. Each possibility represents a separate embodiment. A guide sequence portion can be custom designed to target any desired sequence. Accordingly, a molecule comprising a “guide sequence portion” is a type of targeting molecule. In some embodiments, the guide sequence portion comprises a sequence that is the same as, or differs by no more than 1, 2, 3, 4, or 5 nucleotides from, a guide sequence portion described herein, e.g., a guide sequence set forth in any of SEQ ID NOs: 1-28998. Each possibility represents a separate embodiment. In some of these embodiments, the guide sequence portion comprises a sequence that is the same as a sequence set forth in any of SEQ ID NOs: 1-28998. Throughout this application, the terms “guide molecule,” “RNA guide molecule,” “guide RNA molecule,” and “gRNA molecule” are synonymous with a molecule comprising a guide sequence portion.

The term “non-discriminatory” as used herein refers to a guide sequence portion of an RNA molecule that targets a specific DNA sequence that is common in both alleles of a gene. For example, a non-discriminatory guide sequence portion is capable of targeting both alleles of a gene present in a cell.

In embodiments of the present invention, an RNA molecule comprises a guide sequence portion having 17-50 contiguous nucleotides containing nucleotides in the sequence set forth in any one of SEQ ID NOs: 1-28998. In some embodiments, the guide sequence portion comprises a sequence that is the same as or differs by no more than 1, 2, or 3 nucleotides from a sequence set forth in any of SEQ ID NOs: 1-28998.

The RNA molecule and or the guide sequence portion of the RNA molecule may contain modified nucleotides. Exemplary modifications to nucleotides or polynucleotides may be synthetic and encompass polynucleotides which contain nucleotides comprising bases other than the naturally occurring adenine, cytosine, thymine, uracil, or guanine bases. Modifications to polynucleotides include polynucleotides which contain synthetic, non-naturally occurring nucleosides e.g., locked nucleic acids. Modifications to polynucleotides may be utilized to increase or decrease stability of an RNA. An example of a modified polynucleotide is an mRNA containing 1-methyl pseudo-uridine. For examples of modified polynucleotides and their uses, see U.S. Pat. No. 8,278,036, PCT International Publication No. WO/2015/006747, and Weissman and Kariko (2015), hereby incorporated by reference.

As used herein, “contiguous nucleotides” set forth in a SEQ ID NO refers to nucleotides in a sequence of nucleotides in the order set forth in the SEQ ID NO without any intervening nucleotides.

In embodiments of the present invention, the guide sequence portion may be 25 nucleotides in length and contain 20-22 contiguous nucleotides in the sequence set forth in any one of SEQ ID NOs: 1-28998. In embodiments of the present invention, the guide sequence portion may be less than 22 nucleotides in length. For example, in embodiments of the present invention the guide sequence portion may be 17, 18, 19, 20, or 21 nucleotides in length. In such embodiments the guide sequence portion may consist of 17, 18, 19, 20, or 21 nucleotides, respectively, in the sequence of 17-22 contiguous nucleotides set forth in any one of SEQ ID NOs: 1-28998. For example, a guide sequence portion having 17 nucleotides in the sequence of 17 contiguous nucleotides set forth in SEQ ID NO: 28999 may consist of any one of the following nucleotide sequences (nucleotides excluded from the contiguous sequence are marked in strike-through):

(SEQ ID NO: 28999)
AAAAAAAUGUACUUGGUUCC
17 nucleotide guide sequence 1:
(SEQ ID NO: 29000) [symbol]
AAAAUGUACUUGGUUCC
17 nucleotide guide sequence 2:
(SEQ ID NO: 29001) [symbol]
AAAAAUGUACUUGGUUC [symbol]
17 nucleotide guide sequence 3:
(SEQ ID NO: 29002) [symbol]
AAAAAAUGUACUUGGUU [symbol]
17 nucleotide guide sequence 4:
(SEQ ID NO: 29003)
AAAAAAAUGUACUUGGU

[symbol]

In embodiments of the present invention, the guide sequence portion may be greater than 20 nucleotides in length. For example, in embodiments of the present invention the guide sequence portion may be 21, 22, 23, 24, 25, 26, 27, 28, 29, or 30 nucleotides in length. In such embodiments the guide sequence portion comprises 17-50 nucleotides containing the sequence of 20, 21 or 22 contiguous nucleotides set forth in any one of SEQ ID NOs: 1-28998 and additional nucleotides fully complimentary to a nucleotide or sequence of nucleotides adjacent to the 3′ end of the target sequence, 5′ end of the target sequence, or both.

In embodiments of the present invention a CRISPR nuclease and an RNA molecule comprising a guide sequence portion form a CRISPR complex that binds to a target DNA sequence to effect cleavage of the target DNA sequence. CRISPR nucleases, e.g. Cpf1, may form a CRISPR complex comprising a CRISPR nuclease and RNA molecule without a further tracrRNA molecule. Alternatively, CRISPR nucleases, e.g. Cas9, may form a CRISPR complex between the CRISPR nuclease, an RNA molecule, and a tracrRNA molecule. A guide sequence portion, which comprises a nucleotide sequence that is capable of hybridizing to a specific target DNA sequence, and a sequence portion that participates in CRISPR nuclease binding, e.g. a tracrRNA sequence portion, can be located on the same RNA molecule. Alternatively, a guide sequence portion may be located on one RNA molecule and a sequence portion that participates in CRISPR nuclease binding, e.g. a tracrRNA portion, may located on a separate RNA molecule. A single RNA molecule comprising a guide sequence portion (e.g. a DNA-targeting RNA sequence) and at least one CRISPR protein-binding RNA sequence portion (e.g. a tracrRNA sequence portion), can form a complex with a CRISPR nuclease and serve as the DNA-targeting molecule. In some embodiments, a first RNA molecule comprising a DNA-targeting RNA portion, which includes a guide sequence portion, and a second RNA molecule comprising a CRISPR protein-binding RNA sequence interact by base pairing to form an RNA complex that targets the CRISPR nuclease to a DNA target site or, alternatively, are fused together to form an RNA molecule that complexes with the CRISPR nuclease and targets the CRISPR nuclease to a DNA target site.

In embodiments of the present invention, a RNA molecule comprising a guide sequence portion may further comprise the sequence of a tracrRNA molecule. Such embodiments may be designed as a synthetic fusion of the guide portion of the RNA molecule and the trans-activating crRNA (tracrRNA). (See Jinek et al., 2012). In such an embodiment, the RNA molecule is a single guide RNA (sgRNA) molecule. Embodiments of the present invention may also form CRISPR complexes utilizing a separate tracrRNA molecule and a separate RNA molecule comprising a guide sequence portion. In such embodiments the tracrRNA molecule may hybridize with the RNA molecule via basepairing and may be advantageous in certain applications of the invention described herein.

The term “tracr mate sequence” refers to a sequence sufficiently complementary to a tracrRNA molecule so as to hybridize to the tracrRNA via basepairing and promote the formation of a CRISPR complex. (See U.S. Pat. No. 8,906,616). In embodiments of the present invention, the RNA molecule may further comprise a portion having a tracr mate sequence.

A “gene,” for the purposes of the present disclosure, includes a DNA region encoding a gene product, as well as all DNA regions which regulate the production of the gene product, whether or not such regulatory sequences are adjacent to coding and/or transcribed sequences. Accordingly, a gene includes, but is not necessarily limited to, promoter sequences, terminators, translational regulatory sequences such as ribosome binding sites and internal ribosome entry sites, enhancers, silencers, insulators, boundary elements, replication origins, matrix attachment sites and locus control regions.

“Eukaryotic” cells include, but are not limited to, fungal cells (such as yeast), plant cells, animal cells, mammalian cells and human cells.

The term “nuclease” as used herein refers to an enzyme capable of cleaving the phosphodiester bonds between the nucleotide subunits of nucleic acid. A nuclease may be isolated or derived from a natural source. The natural source may be any living organism. Alternatively, a nuclease may be a modified or a synthetic protein which retains the phosphodiester bond cleaving activity. Gene modification can be achieved using a nuclease, for example a CRISPR nuclease.

According to embodiments of the present invention, there is provided an RNA molecule comprising a guide sequence portion (e.g. a targeting sequence) comprising a nucleotide sequence that is fully or partially complementary to a target located in or near an allele of the B2M gene. In some embodiments, the guide sequence portion of the RNA molecule consists of 16, 17, 18, 19, 20, 21, 22, 23, 24, 25, 26, or more than 26 nucleotides. In some embodiments the guide sequence portion is configured to target a CRISPR nuclease to a B2M target site and induce a double-strand break or a single-strand break within 500, 400, 300, 200, 100, 50, 25, or 10 nucleotides of the B2M target site. In some embodiments, the RNA molecule is a guide RNA molecule such as a crRNA molecule or a single-guide RNA molecule. In some embodiments, the guide sequence portion is complementary to a target sequence located from 30 base pairs upstream to 30 base pairs downstream of Exon 3, Exon 4, Intron 1, Intron 2, Intron 3, or a 3′ untranslated region (3′ UTR) of the B2M gene. In some embodiments, the guide sequence portion is complementary to a target sequence located from 50 base pairs upstream to 50 base pairs downstream of Exon 3, Exon 4, Intron 1, Intron 2, Intron 3, or a 3′ untranslated region (3′ UTR) of the B2M gene. Each possibility represents a separate embodiment. In some embodiments, the guide sequence portion is complementary to a target sequence located from 7 base pairs upstream to 7 base pairs downstream of Exon 3, Exon 4, Intron 1, Intron 2, Intron 3, or a 3′ untranslated region (3′ UTR) of the B2M gene.

As used herein, the term “HSC” refers to both hematopoietic stem cells and hematopoietic stem progenitor cells. Non-limiting examples of stem cells include bone marrow cells, myeloid progenitor cells, a multipotent progenitor cells, and lineage restricted progenitor cells.

As used herein, “progenitor cell” refers to a lineage cell that is derived from stem cell and retains mitotic capacity and multipotency (e.g., can differentiate or develop into more than one but not all types of mature lineage of cell). As used herein “hematopoiesis” or “hemopoiesis” refers to the formation and development of various types of blood cells (e.g., red blood cells, megakaryocytes, myeloid cells (e.g., monocytes, macrophages and neutrophil), and lymphocytes) and other formed elements in the body (e.g., in the bone marrow).

According to some embodiments of the present invention, there is provided a method for modifying in a cell an allele of the Beta-2 microglobulin (B2M) gene, the method comprising
introducing to the cell a composition comprising:
   at least one CRISPR nuclease, or a polynucleotide molecule encoding the CRISPR nuclease; and
   a RNA molecule comprising a guide sequence portion having 17-50 nucleotides, or a nucleotide molecule encoding the same,
wherein a complex of the CRISPR nuclease and the RNA molecule affects a double strand break in the allele of the B2M gene.

In some embodiments, the RNA molecule is a crRNA molecule and the composition further comprises a tracrRNA molecule that forms a crRNA: tracrRNA molecule with the crRNA molecule. In some embodiments, the RNA molecule is an sgRNA molecule.

In some embodiments, the composition also comprises a donor molecule. In some embodiments, a sequence of nucleotides from the donor molecule is inserted or copied at or near the double strand break site. In some embodiments, the composition further comprises a donor molecule comprising a sequence of nucleotides that is introduced at the double strand break site.

In some embodiments, the composition further comprises a donor molecule containing a sequence of nucleotides that is introduced at the double strand break site such that the expression of the introduced sequence is mediated by the promoter of the B2M gene.

In some embodiments, the introduced sequence comprises a sequence from an Alpha-1 antitrypsin, Glucose-6-phosphatase (G6PC), Serpin Family A Member (SERPINA), Transthyretin (TTR), ornithine transcarbamylase, argininosuccinic acid synthetase, arginase, argininosuccinase, carbamoyl phosphate synthetase, and N-acetylglutamate synthetase, Alpha Galactosidase A, Coagulation Factor IX, Coagulation Factor VII, Lysosomal Alpha Glucosidase, Fibrinogen, Phenylalanine 4 Hydroxylase, Alkaline Phosphatase, Glucosylceramidase, Beta Galactosidase, Porphobilinogen Deaminase, Arylsulfatase B, Beta Glucuronidase, Alpha-N-Acetylglucosaminidase, Lysosomal Alpha, Alpha L-Iduronidase, Mannosidase, Phosphatidylcholine Sterol Acyltransferase, N-Sulphoglucosamine Sulphohydrolase, Coagulation Factor X, N-Acetylgalactosamine-6-Sulfatase, Sphingomyelin Phosphodiesterase, iduronate-2-sulfatase, Lysosomal Alpha Glucosidase, Cyclin Dependent Kinase Like 5, Prolow Density Lipoprotein Receptor Related Protein 1, Phenylalanine Ammonia Lyase, Protein Glutamine Gamma Glutamyltransferase K, or Lysosomal Protective Protein encoding gene.

In some embodiments, the introduced sequence comprises a sequence from an acid α-glucosidase, α-L-iduronidase, α-galactosidase, iduronate-2-sulfatase, N-acetylgalactosamine-6-sulfatase, N-acetylgalactosamine-4-sulfatase, a lysophosphatidylcholine metabolism-related protein, preferably phospholipase A2, a T-REC or K-REC related protein, β-glucosidase, β-glucocerebrosidase, arylsulfatase A, Factor VIII, insulin-like growth factor 1 (IGF-1), surfactant protein A, surfactant protein B, aspartyl-β-glucosaminidase, acetyl-CoA α-glucosaminide, acetyl-CoA-arylamine N-acetyltransferase, N-acetylglucosamine-6-sulfatase, N-acetylglucosamine-1-Phosphotransferase, α-N-acetylglucosaminidase, acid ceramidase, aspartacylase, lysosomal acid lipase, acid sphingomyelinase, arylsulfatase B, α-L-fucosidase, galactosylceramidase, galactocerebrosidase, β-galactosidase, protective protein/cathepsin A, β-glucoronidase, heparan N-sulfatase, β-hexosaminidase A, hyaluronidase-1, alpha-D-mannosidase, beta-mannosidase, alpha-neuraminidase, beta-hexosaminidase A, beta-hexosaminidase B, palmitoyl-protein thioesterase, tripeptidyl peptidase I, Battenin, Ceroid-lipofuscinosis neuronal protein 5 (CLN5), Ceroid-lipofuscinosis neuronal protein 6 (CLN6), Ceroid-lipofuscinosis neuronal protein 7 (CLN7), Ceroid-lipofuscinosis neuronal protein 8 (CLN8), (Cathepsin D), cystinosin, cathepsin K, Sialin, Lysosome-associated membrane protein 2 (LAMP2), human growth hormone, follicle-stimulating hormone, erythropoietin, a cytokine, a chemokine, IL-10, IGF1, TGF-β, IL-15, CXCR4, IL-4, or a granulocyte colony-stimulating factor (G-CSF) encoding gene.

In some embodiments, the donor molecule contains a sequence from a gene encoding a protein that is secreted by a cell.

In some embodiments, the introduced sequence comprises a sequence that encodes a polypeptide of interest that is expressed by the cell.

In some embodiments, the expressed polypeptide of interest is secreted by the cell.

In some embodiments, the expressed polypeptide of interest further comprises a signal peptide.

In some embodiments, the signal peptide is encoded by the allele of the B2M gene.

In some embodiments, the introduced sequence comprises a sequence encoding a 2A self-cleaving peptide.

In some embodiments, the introduced sequence comprises a sequence that encodes a signal peptide.

In some embodiments, the signal peptide is a B2M signal peptide.

In some embodiments, the introduced sequence comprises a splice acceptor sequence and a splice donor sequence.

In some embodiments, the introduced sequence comprises a splice acceptor sequence, a sequence encoding a polypeptide of interest, a sequence encoding a 2A self-cleaving peptide, a signal peptide, and a splice donor sequence.

In some embodiments, the donor molecule comprises a first homology arm sequence that shares at least 90%, 90-95%, 95-100%, or preferably 100%, sequence identity with a B2M sequence upstream of the double-strand break and a second homology arm sequence that shares at least 90%, 90-95%, 95-100%, or preferably 100%, sequence identity with a B2M sequence downstream of the double-strand break.

In some embodiments, the first homology arm sequence and second homology arm sequences are each about 20-50, 50-100, 100-200, 200-500, 500-1000, or 1000-2000 nucleotides in length.

In some embodiments, the polypeptide of interest is a soluble protein.

In some embodiments, the polypeptide of interest is about 20-50, 50-100, 100-200, 200-500, 500-1000, or 1000-2000 amino acids in length.

In some embodiments, the RNA molecule comprises a guide sequence portion having 17-50 contiguous nucleotides containing nucleotides in the sequence set forth in any one of SEQ ID NOs: 1-28998.

In some embodiments, the RNA molecule comprises a non-discriminatory guide sequence portion that targets both B2M alleles.

In some embodiments, the RNA molecule comprises a non-discriminatory guide portion that targets any one of Exon 3, Exon 4, Intron 1, Intron 2, Intron 3, or a 3′ untranslated region (3′ UTR) of the B2M gene.

In some embodiments, the RNA molecule comprises a non-discriminatory guide portion that targets a sequence that is located within a genomic range selected from any one of 15:44716329-44716356, 15:44717607-44717813, 15:44717825-44718145, 15:44716357-44717323, 15:44717327-44717606, 15:44711614-44712998, 15:44713001-44713065, 15:44713067-44713182, 15:44713184-44713461, 15:44713463-44714419, 15:44714421-44714474, 15:44714476-44715422, and 15:44715702-44716328.

In some embodiments, the modified allele of the B2M gene expresses a B2M gene product.

In some embodiments, the modified allele of the B2M gene expresses a B2M polypeptide and the polypeptide of interest.

In some embodiments, the cell is a stem cell, a monocyte, a macrophage, an iPS-derived monocyte, an iPS-derived macrophage, a hematopoietic stem cell (HSC), a hematopoietic stem and progenitor cell (HSPC), a myeloid precursor cell, a myeloblast, a lymphoblast, an erythroid precursor cell, a platelet cell, a natural killer (NK) cell, a β-lymphocyte, a T-lymphocyte, an eosinophil, a neutrophil, an iPS-derived cell, or a basophil.

In some embodiments, the cell is a stem cell, and the method further comprises differentiating the stem cell after modifying the stem cell.

According to embodiments of the present invention, there is provided a modified cell obtained by the method of any one of the embodiments presented herein.

According to some embodiments, the cell is a stem cell, a monocyte, a macrophage, an iPS-derived monocyte, an iPS-derived macrophage, a hematopoietic stem cell (HSC), a hematopoietic stem and progenitor cell (HSPC), a myeloid precursor cell, a myeloblast, a lymphoblast, an erythroid precursor cell, a platelet cell, a natural killer (NK) cell, a β-lymphocyte, a T-lymphocyte, an eosinophil, a neutrophil, an iPS-derived cell, or a basophil.

According to embodiments of the present invention, there is provided a RNA molecule comprising a guide sequence portion having 17-50 contiguous nucleotides containing nucleotides in the sequence set forth in any one of SEQ ID NOs: 1-28998.

According to embodiments of the present invention, there is provided a composition comprising the RNA molecule and at least one CRISPR nuclease.

According to some embodiments, the composition further comprises a donor molecule.

In some embodiments, the donor molecule comprises a sequence from an Alpha-1 antitrypsin, Glucose-6-phosphatase (G6PC), Serpin Family A Member (SERPINA), Transthyretin (TTR), ornithine transcarbamylase, argininosuccinic acid synthetase, arginase, argininosuccinase, carbamoyl phosphate synthetase, and N-acetylglutamate synthetase, Alpha Galactosidase A, Coagulation Factor IX, Coagulation Factor VII, Lysosomal Alpha Glucosidase, Fibrinogen, Phenylalanine 4 Hydroxylase, Alkaline Phosphatase, Glucosylceramidase, Beta Galactosidase, Porphobilinogen Deaminase, Arylsulfatase B, Beta Glucuronidase, Alpha-N-Acetylglucosaminidase, Lysosomal Alpha, Alpha L-Iduronidase, Mannosidase, Phosphatidylcholine Sterol Acyltransferase, N-Sulphoglucosamine Sulphohydrolase, Coagulation Factor X, N-Acetylgalactosamine-6-Sulfatase, Sphingomyelin Phosphodiesterase, iduronate-2-sulfatase, Lysosomal Alpha Glucosidase, Cyclin Dependent Kinase Like 5, Prolow Density Lipoprotein Receptor Related Protein 1, Phenylalanine Ammonia Lyase, Protein Glutamine Gamma Glutamyltransferase K, or Lysosomal Protective Protein encoding gene.

In some embodiments, the donor molecule comprises a sequence from an acid α-glucosidase, α-L-iduronidase, α-galactosidase, iduronate-2-sulfatase, N-acetylgalactosamine-6-sulfatase, N-acetylgalactosamine-4-sulfatase, a lysophosphatidylcholine metabolism-related protein, preferably phospholipase A2, a T-REC or K-REC related protein, β-glucosidase, β-glucocerebrosidase, arylsulfatase A, Factor VIII, insulin-like growth factor 1 (IGF-1), surfactant protein A, surfactant protein B, aspartyl-β-glucosaminidase, acetyl-CoA α-glucosaminide, acetyl-CoA-arylamine N-acetyltransferase, N-acetylglucosamine-6-sulfatase, N-acetylglucosamine-1-Phosphotransferase, α-N-acetylglucosaminidase, acid ceramidase, aspartacylase, lysosomal acid lipase, acid sphingomyelinase, arylsulfatase B, α-L-fucosidase, galactosylceramidase, galactocerebrosidase, β-galactosidase, protective protein/cathepsin A, β-glucoronidase, heparan N-sulfatase, β-hexosaminidase A, hyaluronidase-1, alpha-D-mannosidase, beta-mannosidase, alpha-neuraminidase, beta-hexosaminidase A, beta-hexosaminidase B, palmitoyl-protein thioesterase, tripeptidyl peptidase I, Battenin, Ceroid-lipofuscinosis neuronal protein 5 (CLN5), Ceroid-lipofuscinosis neuronal protein 6 (CLN6), Ceroid-lipofuscinosis neuronal protein 7 (CLN7), Ceroid-lipofuscinosis neuronal protein 8 (CLN8), (Cathepsin D), cystinosin, cathepsin K, Sialin, Lysosome-associated membrane protein 2 (LAMP2), human growth hormone, follicle-stimulating hormone, erythropoietin, a cytokine, a chemokine, IL-10, IGF1, TGF-β, IL-15, CXCR4, IL-4, or a granulocyte colony-stimulating factor (G-CSF) encoding gene.

In some embodiments, the donor molecule comprises a sequence from a gene encoding a protein that is secreted by a cell.

In some embodiments, the donor molecule comprises a sequence that encodes a polypeptide of interest. A nucleotide sequence encoding a polypeptide that is desired to be expressed in a target cell and secreted by the cell may be inserted into the B2M allele such that the modified B2M allele is capable of expressing both the polypeptide encoded by the inserted sequence as well as the original B2M gene product.

In some embodiments, the donor molecule comprises a sequence encoding a 2A self-cleaving peptide.

In some embodiments, the donor molecule comprises a sequence that encodes a signal peptide.

In some embodiments, the signal peptide is a B2M signal peptide.

In some embodiments, the donor molecule comprises a splice acceptor sequence and a splice donor sequence.

In some embodiments, the donor molecule comprises a splice acceptor sequence, a sequence encoding a polypeptide of interest, a sequence encoding a 2A self-cleaving peptide, a signal peptide, and a splice donor sequence.

In some embodiments, the donor molecule comprises a first homology arm sequence that shares at least 90%, preferably 100%, sequence identity with a first sequence in the B2M gene, and a second homology arm sequence that shares at least 90%, preferably 100%, sequence identity with a second sequence in the B2M gene.

In some embodiments, the first homology arm sequence and second homology arm sequences are each about 20-50, 50-100, 100-200, 200-500, 500-1000, or 1000-2000 nucleotides in length.

In some embodiments, the polypeptide of interest is a soluble protein.

In some embodiments, the polypeptide of interest is up to 20-50, 50-100, 100-200, 200-500, 500-1000, or 1000-2000 amino acids in length.

According to some embodiments, the composition further comprises a tracrRNA molecule. According to embodiments of the present invention, there is provided a method for modifying or editing a B2M allele in a cell, the method comprising delivering to the cell the composition of any one of the embodiments presented herein.

According to embodiments of the present invention, there is provided use of any one of the compositions presented herein for modifying or editing a B2M allele in a cell, comprising delivering to the cell the composition of any one of the embodiments presented herein.

According to embodiments of the present invention, there is provided a medicament comprising the composition of any one of the embodiments presented herein for use in modifying or editing a B2M allele in a cell, wherein the medicament is administered by delivering to the cell the composition of any one of the embodiments presented herein.

According to embodiments of the present invention, there is provided a kit for modifying or editing a B2M allele in a cell, comprising an RNA molecule of any one of the embodiments presented herein, a CRISPR nuclease, and/or a tracrRNA molecule; and instructions for delivering the RNA molecule; CRISPR nuclease, and/or the tracrRNA to the cell. In some embodiments, the kit further comprises a donor molecule and instructions for delivering the donor molecule to a cell.

According to embodiments of the present invention, there is provided a gene editing composition comprising an RNA molecule comprising a guide sequence portion having 17-50 contiguous nucleotides containing nucleotides in the sequence set forth in any one of SEQ ID NOs: 1-28998. In some embodiments, the RNA molecule further comprises a portion having a sequence which binds to a CRISPR nuclease. In some embodiments, the sequence which binds to a CRISPR nuclease is a tracrRNA sequence. In some embodiments the RNA comprising a guide sequence portion is a crRNA molecule. In some embodiments an RNA molecule comprising a guide sequence portion is a single-guide RNA (sgRNA) molecule.

In some embodiments, the RNA molecule further comprises a portion having a tracr mate sequence.

In some embodiments, the RNA molecule may further comprise one or more linker portions.

According to embodiments of the present invention, an RNA molecule may be up to 1000, 900, 800, 700, 600, 500, 450, 400, 350, 300, 290, 280, 270, 260, 250, 240, 230, 220, 210, 200, 190, 180, 170, 160, 150, 140, 130, 120, 110, or 100 nucleotides in length. Each possibility represents a separate embodiment. In embodiments of the present invention, the RNA molecule may be 17 up to 300 nucleotides in length, 100 up to 300 nucleotides in length, 150 up to 300 nucleotides in length, 100 up to 500 nucleotides in length, 100 up to 400 nucleotides in length, 200 up to 300 nucleotides in length, 100 to 200 nucleotides in length, or 150 up to 250 nucleotides in length. Each possibility represents a separate embodiment.

According to some embodiments of the present invention, the composition further comprises a tracrRNA molecule.

According to some embodiments of the present invention, there is provided a method for modifying or editing a B2M allele in a cell, the method comprising delivering to the cell a composition comprising an RNA molecule comprising a guide sequence portion having 17-50 contiguous nucleotides containing nucleotides in the sequence set forth in any one of SEQ ID NOs: 1-28998 and a CRISPR nuclease. In some embodiments, the composition further comprises a donor molecule.

According to some embodiments of the present invention, there is provided a method for treating a disorder or disease, the method comprising delivering to a cell of a subject having the disorder or disease a composition comprising an RNA molecule comprising a guide sequence portion having 17-50 contiguous nucleotides containing nucleotides in the sequence set forth in any one of SEQ ID NOs: 1-28998 and a CRISPR nuclease. In some embodiments, the composition further comprises a donor molecule.

According to some embodiments of the present invention, there is provided a method for treating a disorder or disease, the method comprising delivering to a cell of a subject having the disorder the composition of any one of the above embodiments or delivering to the subject the modified cell of any one of the above embodiments.

According to some embodiments, the disease or disorder is Pompe disease, Mucopolysaccharidosis disease, Mucopolysaccharidosis type II, Mucopolysaccharidosis type IVA, Mucopolysaccharidosis type VI, Adrenoleukodystrophy, Severe combined immunodeficiency, Gaucher disease, metachromatic leukodystrophy (MLD), primary immune deficiency, hemophilia A, hemophilia B, IGF-1 deficiency, surfactant deficiency, Aspartylglycosaminuria, Sanfilipo syndrome, mucopolysaccharidosis type III, Sanfilippo Syndrome type IIId, I-cell disease, Schindler Disease, Farber disease (FD), Spinal muscular atrophy with progressive myoclonic epilepsy (SMA-PME), Canavan disease, Lysosomal acid lipase deficiency, Niemann-Pick disease, Mucopolysaccharidosis type 6, Fucosidosis, Krabbe disease, GM1 gangliosidosis, mucopolysaccharidosis type IVB (MPS IVB), or galactosialidosis, Sly disease, Mucopolysaccharidosis type III, Late-onset Tay Sachs, Hyaluronidase-1 deficiency, Alpha-mannosidosis, Beta-mannosidosis, Sialidosis, Stanhoff disease, Santavuori-Haltia disease, Jansky-Bielschowsky disease, Batten disease, Neuronal ceroid lipofuscinosis type 5, Neuronal ceroid lipofuscinosis type 6, Neuronal ceroid lipofuscinosis type 7, Neuronal ceroid lipofuscinosis type 8, Congenital cathepsin D deficiency, Cystinosis, Pycnodysostosis, Salla disease, Danon disease, and/or Alpha-1 antitrypsin deficiency.

In some embodiments, the composition or the modified cell is delivered to a tissue or tumor of the subject.

According to some embodiments of the present invention, there is provided a medicament comprising the composition any one of the above embodiments for use in modifying a B2M allele in a cell, wherein the medicament is administered by delivering to the cell the composition any one of the above embodiments.

According to some embodiments of the present invention, there is provided use of the composition any one of the above embodiments or the modified cell of any one of the above embodiments for treating ameliorating or preventing a disorder or disease, comprising delivering to a cell of a subject having or at risk of having the disorder the composition any one of the above embodiments or delivering to the subject the modified cell of any one of the above embodiments.

According to some embodiments of the present invention, there is provided a medicament comprising the composition of any one of the above embodiments or the modified cell of any one of the above embodiments for use in treating ameliorating or preventing a disorder or disease, wherein the medicament is administered by delivering to a cell of a subject having or at risk of having the disorder the composition of any one of the above embodiments or delivering to the subject the modified cell of any one of the above embodiments.

According to some embodiments, the disorder or disease is Pompe disease, Mucopolysaccharidosis Type I, Fabry disease, Mucopolysaccharidosis type II, Mucopolysaccharidosis type IVA, Mucopolysaccharidosis type VI, Adrenoleukodystrophy, Severe combined immunodeficiency, Gaucher disease, metachromatic leukodystrophy (MLD), primary immune deficiency, hemophilia A, hemophilia B, IGF-1 deficiency, surfactant deficiency, Aspartylglycosaminuria, Sanfilipo syndrome, mucopolysaccharidosis type III, Sanfilippo Syndrome type IIId, I-cell disease, Schindler Disease, Farber disease (FD), Spinal muscular atrophy with progressive myoclonic epilepsy (SMA-PME), Canavan disease, Lysosomal acid lipase deficiency, Niemann-Pick disease, Mucopolysaccharidosis type 6, Fucosidosis, Krabbe disease, GM1 gangliosidosis, mucopolysaccharidosis type IVB (MPS IVB), or galactosialidosis, Sly disease, Mucopolysaccharidosis type III, Late-onset Tay Sachs, Hyaluronidase-1 deficiency, Alpha-mannosidosis, Beta-mannosidosis, Sialidosis, Stanhoff disease, Santavuori-Haltia disease, Jansky-Bielschowsky disease, Batten disease, Neuronal ceroid lipofuscinosis type 5, Neuronal ceroid lipofuscinosis type 6, Neuronal ceroid lipofuscinosis type 7, Neuronal ceroid lipofuscinosis type 8, Congenital cathepsin D deficiency, Cystinosis, Pycnodysostosis, Salla disease, Danon disease, and/or Alpha-1 antitrypsin deficiency.

In some embodiments, the disorder or disease is a lysosomal storage disorder.

In some embodiments, the disorder or disease is a disorder or disease of the blood, lungs, brain, liver, guts, intestines, bones, muscles, or central nervous system, or is an inflammatory disease or autoinflammatory disease.

In some embodiments, the composition of any one of the above embodiments or the modified cell of any one of the above embodiments is a medicament that is an enzyme replacement therapy.

According to some embodiments of the present invention, there is provided a method of treating a disease or disorder, wherein the method is an immunotherapy comprising delivering to the subject the modified cell of any one of the above embodiments.

In some embodiments, the disease or disorder is cancer.

In some embodiments, the composition of any one of the above embodiments or the modified cell of any one of the above embodiments is for use in treating ameliorating or preventing a disorder or disease.

According to some embodiments of the present invention, there is provided a method for modifying a DNA target site in a monocyte or macrophage of a subject, wherein the modification of the DNA target site induces the monocyte or macrophage to express a desired protein encoded by the modification, the method comprising delivering to the cell of the subject a composition comprising an RNA molecule comprising a guide sequence portion having 17-50 contiguous nucleotides containing nucleotides in the sequence set forth in any one of SEQ ID NOs: 1-28998 and a CRISPR nuclease. In some embodiments, the composition further comprises a donor molecule.

According to embodiments of the present invention, at least one CRISPR nuclease and the RNA molecule or RNA molecules are delivered to the subject and/or cells substantially at the same time or at different times.

In some embodiments, a tracrRNA molecule is delivered to the subject and/or cells substantially at the same time or at different times as the CRISPR nuclease and RNA molecule or RNA molecules.

According to embodiments of the present invention, the RNA molecule targets an alternative splicing signal sequence between an exon and an intron of a B2M allele.

According to embodiments of the present invention, the RNA molecule is non-discriminatory and targets a sequence present in both B2M alleles. In some embodiments, the sequence is present in both B2M alleles. In some embodiments, the sequence is present in an intron of the B2M gene. In some embodiments, the intron is the first intron that follows the first coding exon of the B2M gene.

Any one of, or combination of, the above-mentioned strategies for modifying or editing a B2M allele may be used in the context of the invention.

In some embodiments, the method comprises contacting at least one allele of a gene of interest with a non-discriminatory RNA molecule, e.g. an RNA molecule comprising a guide sequence portion which is capable of targeting both alleles of a gene, and a CRISPR nuclease e.g., a Cas9 protein, wherein the non-discriminatory RNA molecule and the CRISPR nuclease associate with a nucleotide sequence of the at least one allele of the gene of interest, thereby modifying or editing the at least one allele. Notably, although biallelic cleavage may occur upon introduction of a non-discriminatory RNA molecule to a cell, insertion of a nucleotide sequence at a cleavage site may occur in only a single allele and not in both alleles. Accordingly, inducing biallelic cleavage with a non-discriminatory RNA molecule that targets an intron of the B2M gene may result in preservation of expression of an endogenous B2M gene from one allele and introduction of a nucleotide sequence, e.g. a nucleotide sequence from a donor molecule, in the other allele. Introduction of the nucleotide sequence in a B2M allele may or may not disrupt expression of the B2M encoded gene product at the B2M allele.

In some embodiments, the method comprises contacting an allele of a gene of interest with an RNA molecule and a CRISPR nuclease e.g., a Cas9 protein, wherein the RNA molecule and the CRISPR nuclease associate with a nucleotide sequence of the allele of the gene of interest which differs by at least one nucleotide from a nucleotide sequence of a different allele of the gene of interest, thereby modifying or editing the targeted allele.

In some embodiments, the RNA molecule and a CRISPR nuclease is introduced to a cell encoding the gene of interest. In some embodiments, the cell encoding the gene of interest is in a mammalian subject.

Embodiments of compositions described herein include at least one CRISPR nuclease, RNA molecule(s) comprising a guide sequence portion, and a tracrRNA molecule, which may be separate or attached to an RNA molecule comprising a guide sequence portion, being effective in a subject or cells at the same time. The at least one CRISPR nuclease, RNA molecule(s) comprising a guide sequence portion, and tracrRNA may be delivered substantially at the same time or can be delivered at different times but have effect at the same time. For example, this includes delivering the CRISPR nuclease to the subject or cells before the RNA molecule comprising a guide sequence portion and/or tracrRNA is substantially extant in the subject or cells.

In some embodiments, the cell is a stem cell. In some embodiments, the cell is a monocyte. In some embodiments, the cell is a macrophage. In some embodiments, the cell is an iPS-derived monocyte. In some embodiments, the cell is an iPS-derived macrophage. In some embodiments, the cell is a hematopoietic stem cell (HSC), a hematopoietic stem and progenitor cell (HSPC), a myeloid precursor cell, a myeloblast, a lymphoblast, an erythroid precursor cell, a platelet cell, a natural killer (NK) cell, a β-lymphocyte, a T-lymphocyte, an eosinophil, a neutrophil, an iPS-derived cell, or a basophil.

### Genetic B2M Safe Harbor Knock-Ins to Treat Diseases and Disorders

In some embodiments, methods of the present invention may be used to knock-in a sequence at a B2M safe harbor site. In some embodiments, B2M-mediated expression of the knocked-in sequence is involved in or associated with treatment of a disorder or a disease.

For example, in some embodiments a B2M DNA target site in target cell (e.g. a monocyte, macrophage, a hematopoietic stem cell (HSC), hematopoietic stem and progenitor cells (HSPCs), myeloid precursor cell, myeloblast, lymphoblast, erythroid precursor cell, platelet cell, natural killer (NK) cell, β-lymphocyte, T-lymphocyte, eosinophil, neutrophil, a basophil, or an iPS cell) is modified such that the target cell expresses and secretes a protein product encoded by the modification, e.g. an introduced or knocked-in protein-encoding sequence. These target cells may be utilized, for example, to treat lysosomal storage diseases or other disorders of the blood, lungs, brain, liver, guts, intestines, bones, muscles, or central nervous system, or inflammatory or autoinflammatory diseases. In some embodiments, these modified cells serve as an alternative to traditional enzyme replacement therapies. In some embodiments, these modified cells are used for immunotherapy such as cancer immunotherapy.

As a non-limiting example, expression of the knocked-in sequence may be involved in or associated with treatment of a disease or disorder of the blood, lungs, brain, guts, intestines, bones, liver, muscles, or central nervous system. As non-limiting examples, the knocked in sequence may be a A1AT, G6PC, SERPINA, TTR, ornithine transcarbamylase, argininosuccinic acid synthetase, arginase, argininosuccinase, carbamoyl phosphate synthetase, or N-acetylglutamate synthetase sequence, or a portion thereof.

As a non-limiting example, expression of the knocked-in sequence may be involved in or associated with treatment of an inflammatory or autoinflammatory disease or disorder, such as inflammatory bowel disease (IBD). For example, IL-4 insertion into neuronal cells shows reduction in symptoms of multiple sclerosis mice. Furthermore, insertion of IL-10 into a type-1-diabetes mouse model exhibits reduction of insulitis and reduced T cell activation. As non-limiting examples, the knocked-in sequence may be a cytokine or a chemokine such as anti-inflammatory cytokines or chemokines (e.g., IL-10, IGF1, TGF-β and IL-4). In such embodiments, the secretion of the knocked-in cytokine or chemokine facilitates the manipulation of immune and inflammatory responses for the treatment of disease or disorder.

As a non-limiting example, expression of the knocked-in sequence may be involved in or associated with treatment of cancer. As non-limiting examples, the knocked in sequence may be a cytokine, a chemokine, a factor, or a protein capable of activating the immune system or helping to recruit immune system cells to the tumor site to eliminate the tumor. For example, IL-15 may be knocked-in and secreted to increase persistence of T cells or natural killer cells, or CXCR4 may be knocked-in to increase recruitment of T cells and natural killer cells to a tumor site.

As a non-limiting example, a target cell (e.g. a monocyte or macrophage) may be modified to express a cytokine or a chemokine, including but limited to IL-10, IGF1, TGF-β, IL-15, CXCR4 and/or IL-4.

As a non-limiting example, expression of the knocked-in sequence may be involved in or associated with treatment of a lysosomal storage disease or other disorder. As a non-limiting example, a target cell (e.g. a monocyte or macrophage) may be modified to express Alpha Galactosidase A, Coagulation Factor IX, Coagulation Factor VII, Lysosomal Alpha Glucosidase, Fibrinogen, Phenylalanine 4 Hydroxylase, Alkaline Phosphatase, Glucosylceramidase, Beta Galactosidase, Porphobilinogen Deaminase, Arylsulfatase B, Beta Glucuronidase, Alpha-N-Acetylglucosaminidase, Lysosomal Alpha, Alpha L-Iduronidase, Mannosidase, Phosphatidylcholine Sterol Acyltransferase, N-Sulphoglucosamine Sulphohydrolase, Coagulation Factor X, N-Acetylgalactosamine-6-Sulfatase, Sphingomyelin Phosphodiesterase, Alpha-1 antitrypsin, iduronate-2-sulfatase, Lysosomal Alpha Glucosidase, Cyclin Dependent Kinase Like 5, Prolow Density Lipoprotein Receptor Related Protein 1, Phenylalanine Ammonia Lyase, Protein Glutamine Gamma Glutamyltransferase K, Lysosomal Protective Protein, or a portion thereof.

As non-limiting examples, expression of the knocked-in sequence may be involved in or associated with treatment of any one of the following diseases or disorders (which are each followed by a related gene or enzyme in parentheses): Pompe disease (acid α-glucosidase), Mucopolysaccharidosis Type I (α-L-iduronidase), Fabry disease (α-galactosidase), Mucopolysaccharidosis type II (iduronate-2-sulfatase), Mucopolysaccharidosis type IVA (N-acetylgalactosamine-6-sulfatase), Mucopolysaccharidosis type VI (N-acetylgalactosamine-4-sulfatase), Adrenoleukodystrophy (a lysophosphatidylcholine metabolism-related gene, e.g. phospholipase A2), Severe combined immunodeficiency (T-REC or K-REC related gene), Gaucher disease (β-glucosidase or β-glucocerebrosidase), metachromatic leukodystrophy (MLD) (arylsulfatase A), primary immune deficiency, hemophilia A and B (Factor VIII), IGF-1 deficiency (IGF-1), surfactant deficiency (surfactant protein A (SP-A) and/or surfactant protein B (SP-B), Aspartylglycosaminuria (aspartyl-β-glucosaminidase), Sanfilipo syndrome (acetyl-CoA α-glucosaminide), mucopolysaccharidosis type III (acetyl-CoA-arylamine N-acetyltransferase), Sanfilippo Syndrome type IIId (N-acetylglucosamine-6-sulfatase), I-cell disease (N-acetylglucosamine-1-Phosphotransferase), Schindler Disease (α-N-acetylglucosaminidase), Farber disease (FD) or Spinal muscular atrophy with progressive myoclonic epilepsy (SMA-PME) (acid ceramidase), Canavan disease (aspartacylase), Lysosomal acid lipase deficiency (lysosomal acid lipase), Niemann-Pick disease (acid sphingomyelinase), Mucopolysaccharidosis type 6 (arylsulfatase B), Fucosidosis (α-L-fucosidase), Krabbe disease (galactosylceramidase, galactocerebrosidase), GM1 gangliosidosis, mucopolysaccharidosis type IVB (MPS IVB), or galactosialidosis (galactasidase-beta-1 or β-galactosidase (GLB1), or protective protein/cathepsin A), Sly disease (β-glucoronidase), Mucopolysaccharidosis type III (heparan N-sulfatase), Late-onset Tay Sachs (β-hexosaminidase A), Hyaluronidase-1 deficiency (hyaluronidase-1), Alpha-mannosidosis (alpha-D-mannosidase), Beta-mannosidosis (beta-mannosidase), Sialidosis (alpha-neuraminidase), Stanhoff disease (beta-hexosaminidase A and/or beta-hexosaminidase B), Santavuori-Haltia disease (palmitoyl-protein thioesterase), Jansky-Bielschowsky disease (tripeptidyl peptidase I), Batten disease (Battenin), Neuronal ceroid lipofuscinosis type 5 (Ceroid-lipofuscinosis neuronal protein 5 (CLN5)), Neuronal ceroid lipofuscinosis type 6 (CLN6), Neuronal ceroid lipofuscinosis type 7 (CLN7), Neuronal ceroid lipofuscinosis type 8 (CLN8), Congenital cathepsin D deficiency (Cathepsin D), Cystinosis (cystinosin), Pycnodysostosis (cathepsin K), Salla disease (Sialin), and/or Danon disease (Lysosome-associated membrane protein 2 (LAMP2)). As a non-limiting example, a target cell (e.g. a monocyte or macrophage) may be modified to express a metabolic modulator. As a non-limiting example, a target cell may be modified to express human growth hormone, insulin-like growth factor 1 (IGF-1), Factor VIII (hemophilia A and B), follicle-stimulating hormone, erythropoietin, a cytokine, a chemokine, IL-10, IGF1, TGF-β, IL-15, CXCR4, IL-4, granulocyte colony-stimulating factor (G-CSF), galactosamine-6-sulfatase, and/or β-hexosamini enzymes.

Specifically, monocytes and macrophages reside in target tissues, including in the lungs and brain, and can serve as an expression vector for long-term secretion of proteins in those target tissues. Expression of a transgene under the control of a B2M promoter is achieved by CRISPR mediated knock-in at a safe harbor site that is targeted by the guide sequence portions described herein. The protein product of the expressed transgene may be secreted. Accordingly, the ability to generate blood and/or tissues containing modified target cells which continuously secrete a protein of interest serves as an alternative to enzyme replacement therapy (ERT). For example, modified monocytes may be useful to target the central nervous system (CNS) or the lungs and secrete a protein of interest in the target tissue.

Such an approach is also useful, for example, to treat lysosomal storage diseases. Some of these diseases also display a phenotype in the CNS. Notably, there is a challenge to treat brain damage with enzyme replacement therapy due to the brain blood barrier (BBB). Monocytes may be utilized for delivery across the BBB such that the secreted protein of interest will be secreted in the CNS.

For example, modified monocytes or macrophages delivered to the brain may be utilized to treat a lysosomal storage disease; modified monocytes or macrophages delivered to the lungs may be utilized to treat anti-trypsin deficiency (A1AT), D-Surfactant deficiency, or proteinosis; or modified monocytes or macrophages in the blood may be utilized to treat A1AT or deficiency of adenosine deaminase 2 (DADA2).

B2M editing strategies include strategies that enable expression of a desired sequence under the control of a B2M promoter, optionally without knocking out the edited B2M alleles. This can be achieved by the following strategies: (1) knock-in in the first intron of a B2M allele, or the first intron that follows the first coding exon of the B2M allele or (2) knock-in by replacement of the stop codon of a B2M allele.

For the first strategy (i.e. utilizing a RNA molecule comprising guide sequence portion targeting Intron 1 of the B2M gene and mediating a biallelic break), since the break is mediated in a nonregulatory region, it is not expected to affect the expression of the B2M gene. For knock-in of a desired sequence without knocking-out the endogenous B2M gene, the desired sequence will be inserted as a new exon (e.g. a new exon 2), by adding a splicing acceptor (SA), a branch site, and a splicing donor (SD) element to the knock-in cassette (e.g. as part of a donor molecule). In this manner a mRNA comprising two coding regions (denoted a “bicistronic transcript”) is generated. In addition, to prevent the interruption of the expression of the endogenous B2M gene, the cassette will include a self-cleaving peptide (e.g., 2A self-cleaving peptide such as P2A, F2A, E2A, T2A, or combination of two or more thereof) and/or the signal peptide of B2M at the C-terminus of the inserted gene, which will be cleaved in the endoplasmic reticulum enabling the separation of the inserted gene product from the B2M gene product.

For the second strategy (i.e. knock-in by replacing the B2M stop codon), the biallelic break will be mediated in the 3′UTR region up to 150 nucleotides downstream to the stop codon or upstream to the stop codon but within an intron region. The location of the break is not expected to affect the expression of the B2M gene. The knocked-in desired sequence will be inserted instead of the stop codon and the knock-in cassette will contain a 2A self-cleaving peptide at the N-terminus of the inserted sequence enabling the separation of the inserted gene product from the B2M gene product.

B2M editing strategies may be utilized to edit T cells for use in chimeric antigen receptor-T cell (CAR-T) therapies. For example, a sequence of interest encoded by a donor molecule (e.g. a CD-19 sequence) may be introduced to a B2M safe harbor site that is targeted by an RNA molecule comprising a guide sequence portion described herein. Such editing may be utilized to alter the T-cell receptor expression and/or receptor signaling exhibited by the cell.

### CRISPR Nucleases and PAM Recognition

In some embodiments, the sequence specific nuclease is selected from CRISPR nucleases, or a functional variant thereof. In some embodiments, the sequence specific nuclease is an RNA-guided DNA nuclease. In some embodiments, the CRISPR complex does not further comprise a tracrRNA. In a non-limiting example, in which the RNA-guided DNA nuclease is a CRISPR protein, the at least one nucleotide which differs between B2M alleles may be within the PAM site and/or proximal to the PAM site within the region that the RNA molecule is designed to hybridize to. A skilled artisan will appreciate that RNA molecules can be engineered to bind to a target of choice in a genome by commonly known methods in the art.

The term “PAM” as used herein refers to a nucleotide sequence of a target DNA located in proximity to the targeted DNA sequence and recognized by the CRISPR nuclease complex. The PAM sequence may differ depending on the nuclease identity. In addition, there are CRISPR nucleases that can target almost all PAMs. In some embodiments of the present invention, a CRISPR system utilizes one or more RNA molecules having a guide sequence portion to direct a CRISPR nuclease to a target DNA site via Watson-Crick base-pairing between the guide sequence portion and the protospacer on the target DNA site, which is next to the protospacer adjacent motif (PAM), which is an additional requirement for target recognition. The CRISPR nuclease then mediates cleavage of the target DNA site to create a double-stranded break within the protospacer. In a non-limiting example, a type II CRISPR system utilizes a mature crRNA: tracrRNA complex that directs the CRISPR nuclease, e.g. Cas9 to the target DNA the target DNA via Watson-Crick base-pairing between the guide sequence portion of the crRNA and the protospacer on the target DNA next to the protospacer adjacent motif (PAM). A skilled artisan will appreciate that each of the engineered RNA molecule of the present invention is further designed such as to associate with a target genomic DNA sequence of interest next to a protospacer adjacent motif (PAM), e.g., a PAM matching the sequence relevant for the type of CRISPR nuclease utilized, such as for a non-limiting example, NGG or NAG, wherein “N” is any nucleobase, for Streptococcus pyogenes Cas9 WT (SpCAS9); NNGRRT for Staphylococcus aureus (SaCas9); NNNVRYM for Jejuni Cas9 WT; NGAN or NGNG for SpCas9-VQR variant; NGCG for SpCas9-VRER variant; NGAG for SpCas9-EQR variant; NRRH for SpCas9-NRRH variant, wherein N is any nucleobase, R is A or G and His A, C, or T; NRTH for SpCas9-NRTH variant, wherein N is any nucleobase, R is A or G and H is A, C, or T; NRCH for SpCas9-NRCH variant, wherein N is any nucleobase, R is A or G and His A, C, or T; NG for SpG variant of SpCas9 wherein N is any nucleobase; NG or NA for SpCas9-NG variant of SpCas9 wherein N is any nucleobase; NR or NRN or NYN for SpRY variant of SpCas9, wherein N is any nucleobase, R is A or G and Y is C or T; NNG for Streptococcus canis Cas9 variant (ScCas9), wherein N is any nucleobase; NNNRRT for SaKKH-Cas9 variant of Staphylococcus aureus (SaCas9), wherein N is any nucleobase, and R is A or G; NNNNGATT for Neisseria meningitidis (NmCas9), wherein N is any nucleobase; TTN for Alicyclobacillus acidiphilus Cas12b (AacCas12b), wherein N is any nucleobase; or TTTV for Cpf1, wherein V is A, C or G. RNA molecules of the present invention are each designed to form complexes in conjunction with one or more different CRISPR nucleases and designed to target polynucleotide sequences of interest utilizing one or more different PAM sequences respective to the CRISPR nuclease utilized.

In some embodiments, an RNA-guided DNA nuclease e.g., a CRISPR nuclease, may be used to cause a DNA break, either double or single-stranded in nature, at a desired location in the genome of a cell. The most commonly used RNA-guided DNA nucleases are derived from CRISPR systems, however, other RNA-guided DNA nucleases are also contemplated for use in the genome editing compositions and methods described herein. For instance, see U.S. Publication No. 2015/0211023, incorporated herein by reference.

CRISPR systems that may be used in the practice of the invention vary greatly. CRISPR systems can be a type I, a type II, or a type III system. Non-limiting examples of suitable CRISPR proteins include Cas3, Cas4, Cas5, Cas5e (or CasD), Cas6, Cas6e, Cas6f, Cas7, Cas8a1, Cas8a2, Cas8b, Cas8c, Cas9, Cas10, Cas1 Od, CasF, CasG, CasH, Csy1, Csy2, Csy3, Cse1 (or CasA), Cse2 (or CasB), Cse3 (or CasE), Cse4 (or CasC), Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csz1, Csx15, Csf1, Csf2, Csf3, Csf4, and Cul966.

In some embodiments, the RNA-guided DNA nuclease is a CRISPR nuclease derived from a type II CRISPR system (e.g., Cas9). The CRISPR nuclease may be derived from Streptococcus pyogenes, Streptococcus thermophilus, Streptococcus sp., Staphylococcus aureus, Neisseria meningitidis, Treponema denticola, Nocardiopsis dassonvillei, Streptomyces pristinaespiralis, Streptomyces viridochromogenes, Streptomyces viridochromogenes, Streptosporangium roseum, Streptosporangium roseum, Alicyclobacillus acidocaldarius, Bacillus pseudomycoides, Bacillus selenitireducens, Exiguobacterium sibiricum, Lactobacillus delbrueckii, Lactobacillus salivarius, Microscilla marina, Burkholderiales bacterium, Polaromonas naphthalenivorans, Polaromonas sp., Crocosphaera watsonii, Cyanothece sp., Microcystis aeruginosa, Synechococcus sp., Acetohalobium arabaticum, Ammonifex degensii, Caldicelulosiruptor becscii, Candidatus desulforudis, Clostridium botulinum, Clostridium difficile, Finegoldia magna, Natranaerobius thermophilus, Pelotomaculum thermopropionicum, Acidithiobacillus caldus, Acidithiobacillus ferrooxidans, Allochromatium vinosum, Marinobacter sp., Nitrosococcus halophilus, Nitrosococcus watsoni, Pseudoalteromonas haloplanktis, Ktedonobacter racemifer, Methanohalobium evestigatum, Anabaena variabilis, Nodularia spumigena, Nostoc sp., Arthrospira maxima, Arthrospira platensis, Arthrospira sp., Lyngbya sp., Microcoleus chthonoplastes, Oscillatoria sp., Petrotoga mobilis, Thermosipho africanus, Acaryochloris marina, or any species which encodes a CRISPR nuclease with a known PAM sequence. CRISPR nucleases encoded by uncultured bacteria may also be used in the context of the invention. (See Burstein et al. Nature, 2017). Variants of CRISPR proteins having known PAM sequences e.g., SpCas9 D1135E variant, SpCas9 VQR variant, SpCas9 EQR variant, or SpCas9 VRER variant may also be used in the context of the invention.

Thus, an RNA-guided DNA nuclease of a CRISPR system, such as a Cas9 protein or modified Cas9 or homolog or ortholog of Cas9, or other RNA-guided DNA nucleases belonging to other types of CRISPR systems, such as Cpf1 and its homologs and orthologs, may be used in the compositions of the present invention. Additional CRISPR nucleases may also be used, for example, the nucleases described in PCT International Application Publication Nos. WO2020/223514 and WO2020/223553, which are hereby incorporated by reference

In certain embodiments, the CRISPR nuclease may be a “functional derivative” of a naturally occurring Cas protein. A “functional derivative” of a native sequence polypeptide is a compound having a qualitative biological property in common with a native sequence polypeptide. “Functional derivatives” include, but are not limited to, fragments of a native sequence and derivatives of a native sequence polypeptide and its fragments, provided that they have a biological activity in common with a corresponding native sequence polypeptide. A biological activity contemplated herein is the ability of the functional derivative to hydrolyze a DNA substrate into fragments. The term “derivative” encompasses both amino acid sequence variants of polypeptide, covalent modifications, and fusions thereof. Suitable derivatives of a Cas polypeptide or a fragment thereof include but are not limited to mutants, fusions, covalent modifications of Cas protein or a fragment thereof. Derivatives include, but are not limited to, CRISPR nickases, catalytically inactive or “dead” CRISPR nucleases, and fusion of a CRISPR nuclease or derivative thereof to other enzymes such as base editors or retrotransposons. See for example, Anzalone et al. (2019) and PCT International Application No. PCT/US2020/037560.

In some embodiments, the CRISPR nuclease or derivative thereof may be fused to a protein that has an enzymatic activity. In some embodiments, the enzymatic activity modifies a target DNA. In some embodiments, the enzymatic activity is nuclease activity, methyltransferase activity, demethylase activity, DNA repair activity, DNA damage activity, deamination activity, dismutase activity, alkylation activity, depurination activity, oxidation activity, pyrimidine dimer forming activity, integrase activity, transposase activity, recombinase activity, polymerase activity, ligase activity, helicase activity, photolyase activity or glycosylase activity. In some cases, the enzymatic activity is nuclease activity. In some cases, the nuclease activity introduces a double strand break in the target DNA. In some cases, the enzymatic activity modifies a target polypeptide associated with the target DNA. In some cases, the enzymatic activity is methyltransferase activity, demethylase activity, acetyltransferase activity, deacetylase activity, kinase activity, phosphatase activity, ubiquitin ligase activity, deubiquitinating activity, adenylation activity, deadenylation activity, SUMOylating activity, deSUMOylating activity, ribosylation activity, deribosylation activity, myristoylation activity or demyristoylation activity. In some cases, the target polypeptide is a histone and the enzymatic activity is methyltransferase activity, demethylase activity, acetyltransferase activity, deacetylase activity, kinase activity, phosphatase activity, ubiquitin ligase activity or deubiquitinating activity.

Cas protein, which includes Cas protein or a fragment thereof, as well as derivatives of Cas protein or a fragment thereof, may be obtainable from a cell or synthesized chemically or by a combination of these two procedures. The cell may be a cell that naturally produces Cas protein, or a cell that naturally produces Cas protein and is genetically engineered to produce the endogenous Cas protein at a higher expression level or to produce a Cas protein from an exogenously introduced nucleic acid, which nucleic acid encodes a Cas that is same or different from the endogenous Cas. In some cases, the cell does not naturally produce Cas protein and is genetically engineered to produce a Cas protein.

In some embodiments, the CRISPR nuclease is Cpf1. Cpf1 is a single RNA-guided endonuclease which utilizes a T-rich protospacer-adjacent motif. Cpf1 cleaves DNA via a staggered DNA double-stranded break. Two Cpf1 enzymes from Acidaminococcus and Lachnospiraceae have been shown to carry out efficient genome-editing activity in human cells. (See Zetsche et al., 2015).

Thus, an RNA-guided DNA nuclease of a Type II CRISPR System, such as a Cas9 protein or modified Cas9 or homologs, orthologues, or variants of Cas9, or other RNA-guided DNA nucleases belonging to other types of CRISPR systems, such as Cpf1 and its homologs, orthologues, or variants, may be used in the present invention.

In some embodiments, the guide molecule comprises one or more chemical modifications which imparts a new or improved property (e.g., improved stability from degradation, improved hybridization energetics, or improved binding properties with an RNA-guided DNA nuclease). Suitable chemical modifications include, but are not limited to: modified bases, modified sugar moieties, or modified inter-nucleoside linkages. Non-limiting examples of suitable chemical modifications include: 4-acetylcytidine, 5-(carboxyhydroxymethyl) uridine, 2′-O-methylcytidine, S-carboxymethylaminomethyl-2-thiouridine, 5-carboxymethylaminomethyluridine, dihydrouridine, 2′-O-methylpseudouridine, “beta, D-galactosylqueuosine”, 2′-O-methylguanosine, inosine, N6-isopentenyladenosine, 1-methyladenosine, 1-methylpseudouridine, 1-methylguanosine, 1-methylinosine, “2,2-dimethylguanosine”, 2-methyladenosine, 2-methylguanosine, 3-methylcytidine, 5-methylcytidine, N6-methyladenosine, 7-methylguanosine, 5-methylaminomethyluridine, 5-methoxyaminomethyl-2-thiouridine, “beta, D-mannosylqueuosine”, 5-methoxycarbonylmethyl-2-thiouridine, 5-methoxycarbonylmethyluridine, 5-methoxyuridine, 2-methylthio-N6-isopentenyladenosine, N-((9-beta-D-ribofuranosyl-2-methylthiopurine-6-yl)carbamoyl) threonine, N-((9-beta-D-ribofuranosylpurine-6-yl)N-methylcarbamoyl) threonine, uridine-5-oxyacetic acid-methylester, uridine-5-oxyacetic acid, wybutoxosine, queuosine, 2-thiocytidine, S-methyl-2-thiouridine, 2-thiouridine, 4-thiouridine, 5-methyluridine, N-((9-beta-D-ribofuranosylpurine-6-yl)-carbamoyl) threonine, 2′-O-methyl-5-methyluridine, 2′-O-methyluridine, wybutosine, “3-(3-amino-3-carboxy-propyl) uridine, (acp3)u”, 2′-O-methyl (M), 3′-phosphorothioate (MS), 3′-thioPACE (MSP), pseudouridine, or 1-methyl pseudo-uridine. Each possibility represents a separate embodiment of the present invention.

Guide Sequences which Target a B2M Allele

Any given RNA molecule comprising a guide sequence portion utilized to target a DNA site may result in degradation of the RNA molecule, limited activity, no activity, or off-target effects. Accordingly, suitable guide sequence portions are necessary for targeting a given DNA site in a gene.

By the present invention, a novel set of guide sequence portions have been identified for targeting at least one B2M allele and introducing to the at least one allele a sequence of nucleotides to be expressed under the control of the B2M promoter. Such a gene editing approach may be used to treat a disorder or disease or modify behavior of a cell. Preferably, a non-discriminatory RNA molecule capable of targeting both B2M alleles is used for targeting.

In some embodiments of the present invention, an RNA molecule is used to target a site in the B2M gene to introduce, or knock-in, an exogenous sequence of nucleotides into the B2M gene. In some embodiments, the location of the site is near the intended knock-in site, preferably near the start codon or the stop codon, preferably within 150 nucleotides of the start codon or stop codon. In some embodiments, the site is located within the first intron that follows the first coding exon of a targeted B2M allele. In some embodiments, the site is located within Exon 3, Exon 4, Intron 1, Intron 2, Intron 3, or a 3′ untranslated region (3′ UTR) of a targeted B2M allele.

### Delivery to Cells

The compositions described herein may be delivered to a target cell by any suitable means. Compositions of the present invention may be targeted to any cell which contains and/or expresses a B2M allele, including any mammalian cell, preferably a monocyte or macrophage. For example, in one embodiment an RNA molecule that specifically targets a B2M allele is delivered to a target cell and the target cell is monocyte or macrophage. The delivery to the cell may be performed in vitro, ex vivo, or in vivo. Further, the nucleic acid compositions described herein may be delivered as one or more of DNA molecules, RNA molecules, ribonucleoproteins (RNPs), nucleic acid vectors, or any combination thereof.

In some embodiments, in vivo delivery methods of the compositions described herein include delivery by a lentivirus, adeno-associated virus (AAV) or nanoparticle. In some embodiments, in vivo delivery methods of the compositions described herein include delivery by a lentivirus, adeno-associated virus (AAV) or nanoparticle. The composition may be in the form of an RNP composition. Accordingly, the delivery can be in vivo to monocytes or macrophages within a subject.

In some embodiments, any one of the compositions described herein is delivered to a cell ex vivo. In some embodiments, the cell is a stem cell. In some embodiments, the cell is a monocyte. In some embodiments, the cell is a macrophage. In some embodiments, the cell is an iPS-derived monocyte or macrophage. In some embodiments, the cell is a hematopoietic stem cell (HSC), a hematopoietic stem and progenitor cell (HSPC), a myeloid precursor cell, a myeloblast, a lymphoblast, an erythroid precursor cell, a platelet cell, a natural killer (NK) cell, a β-lymphocyte, a T-lymphocyte, an eosinophil, a neutrophil, or a basophil The composition may be delivered to the cell by any known ex vivo delivery method, including but not limited to, electroporation, viral transduction, nanoparticle delivery, liposomes, etc. The composition may be in the form of an RNP composition. Additional detailed delivery methods are described throughout this section.

In some embodiments, an RNA molecule of a composition described herein comprises a chemical modification. Non-limiting examples of suitable chemical modifications include 2′-0-methyl (M), 2′-0-methyl, 3′phosphorothioate (MS) or 2′-0-methyl, 3′thioPACE (MSP), pseudouridine, and 1-methyl pseudo-uridine. Each possibility represents a separate embodiment of the present invention.

Any suitable viral vector system may be used to deliver nucleic acid compositions e.g., the RNA molecule compositions of the subject invention. Conventional viral and non-viral based gene transfer methods can be used to introduce nucleic acids and target tissues. In certain embodiments, nucleic acids are administered for in vivo or ex vivo gene therapy uses. Non-viral vector delivery systems include naked nucleic acid, and nucleic acid complexed with a delivery vehicle such as a liposome or poloxamer. For a review of gene therapy procedures, see Anderson (1992); Nabel & Felgner (1993); Mitani & Caskey (1993); Dillon (1993); Miller (1992); Van Brunt (1988); Vigne (1995); Kremer & Perricaudet (1995); Haddada et al. (1995); and Yu et al. (1994).

Methods of non-viral delivery of nucleic acids and/or proteins include electroporation, lipofection, microinjection, biolistics, particle gun acceleration, virosomes, liposomes, immunoliposomes, lipid nanoparticles (LNPs), polycation or lipid:nucleic acid conjugates, artificial virions, and agent-enhanced uptake of nucleic acids or can be delivered to plant cells by bacteria or viruses (e.g., Agrobacterium, Rhizobium sp. NGR234, Sinorhizobium meliloti, Mesorhizobium loti, tobacco mosaic virus, potato virus X, cauliflower mosaic virus and cassava vein mosaic virus). (See, e.g., Chung et al., 2006). Sonoporation using, e.g., the Sonitron 2000 system (Rich-Mar), can also be used for delivery of nucleic acids. Cationic-lipid mediated delivery of proteins and/or nucleic acids is also contemplated as an in vivo, ex vivo, or in vitro delivery method. (See Zuris et al. (2015); see also Coelho et al. (2013); Judge et al. (2006); and Basha et al. (2011)).

Non-viral vectors, such as transposon-based systems e.g. recombinant Sleeping Beauty transposon systems or recombinant PiggyBac transposon systems, may also be delivered to a target cell and utilized for transposition of a polynucleotide sequence of a molecule of the composition or a polynucleotide sequence encoding a molecule of the composition in the target cell.

Additional exemplary nucleic acid delivery systems include those provided by Amaxa® Biosystems (Cologne, Germany), Maxcyte, Inc. (Rockville, Md.), BTX Molecular Delivery Systems (Holliston, Mass.) and Copernicus Therapeutics Inc., (see, e.g., U.S. Pat. No. 6,008,336). Lipofection is described in e.g., U.S. Pat. Nos. 5,049,386, 4,946,787; and 4,897,355, and lipofection reagents are sold commercially (e.g., Transfectam™, Lipofectin™ and Lipofectamine™ RNAiMAX). Cationic and neutral lipids that are suitable for efficient receptor-recognition lipofection of polynucleotides include those disclosed in PCT International Publication Nos. WO/1991/017424 and WO/1991/016024. Delivery can be to cells (ex vivo administration) or target tissues (in vivo administration).

The preparation of lipid:nucleic acid complexes, including targeted liposomes such as immunolipid complexes, is well known to one of skill in the art (see, e.g., Crystal, Science (1995); Blaese et al., (1995); Behr et al., (1994); Remy et al. (1994); Gao and Huang (1995); Ahmad and Allen (1992); U.S. Pat. Nos. 4,186,183; 4,217,344; 4,235,871; 4,261,975; 4,485,054; 4,501,728; 4,774,085; 4,837,028; and 4,946,787).

Additional methods of delivery include the use of packaging the nucleic acids to be delivered into EnGeneIC delivery vehicles (EDVs). These EDVs are specifically delivered to target tissues using bispecific antibodies where one arm of the antibody has specificity for the target tissue and the other has specificity for the EDV. The antibody brings the EDVs to the target cell surface and then the EDV is brought into the cell by endocytosis. Once in the cell, the contents are released (See MacDiarmid et al., 2009).

Delivery vehicles include, but are not limited to, bacteria, preferably non-pathogenic, vehicles, nanoparticles, exosomes, microvesicles, gene gun delivery, for example, by attachment of a composition to a gold particle which is fired into a cell using via a “gene-gun”, viral vehicles, including but not limited to lentiviruses, AAV, and retroviruses), virus-like particles (VLPs). large VLPs (LVLPs), lentivirus-like particles, transposons, viral vectors, naked vectors, DNA, or RNA, among other delivery vehicles known in the art.

The delivery of a CRISPR nuclease and/or a polynucleotide encoding the CRISPR nuclease, and optionally additional nucleotide molecules and/or additional proteins or peptides, may be performed by utilizing a single delivery vehicle or method or a combination of different delivery vehicles or methods. For example, a CRISPR nuclease may be delivered to a cell utilizing an LNP, and a crRNA molecule and tracrRNA molecule may be delivered to the cell utilizing AAV. Alternatively, a CRISPR nuclease may be delivered to a cell utilizing an AAV particle, and a crRNA molecule and tracrRNA molecule may be delivered to the cell utilizing a separate AAV particle, which may be advantageous due to size limitations.

The use of RNA or DNA viral based systems for viral mediated delivery of nucleic acids take advantage of highly evolved processes for targeting a virus to specific cells in the body and trafficking the viral payload to the nucleus. Viral vectors can be administered directly to patients (in vivo) or they can be used to treat cells in vitro and the modified cells are administered to patients (ex vivo). Conventional viral based systems for the delivery of nucleic acids include, but are not limited to, retroviral, lentivirus, adenoviral, adeno-associated, vaccinia and herpes simplex virus vectors for gene transfer. An RNA virus may be utilized for delivery of the RNA compositions described herein. Additionally, high transduction efficiencies have been observed in many different cell types and target tissues. Nucleic acid of the invention may be delivered by non-integrating lentivirus. Optionally, RNA delivery with Lentivirus is utilized. Optionally the lentivirus includes mRNA of the nuclease, RNA of the guide. Optionally the lentivirus includes mRNA of the nuclease, RNA of the guide and a donor template. Optionally, the lentivirus includes the nuclease protein, guide RNA. Optionally, the lentivirus includes the nuclease protein, guide RNA and/or a donor template for gene editing via, for example, homology directed repair. Optionally the lentivirus includes mRNA of the nuclease, DNA-targeting RNA, and the tracrRNA. Optionally the lentivirus includes mRNA of the nuclease, DNA-targeting RNA, and the tracrRNA, and a donor template. Optionally, the lentivirus includes the nuclease protein, DNA-targeting RNA, and the tracrRNA. Optionally, the lentivirus includes the nuclease protein, DNA-targeting RNA, and the tracrRNA, and a donor template for gene editing via, for example, homology directed repair.

The tropism of a retrovirus can be altered by incorporating foreign envelope proteins, expanding the potential target population of target cells. Lentiviral vectors are retroviral vectors that are able to transduce or infect non-dividing cells and typically produce high viral titers. Selection of a retroviral gene transfer system depends on the target tissue. Retroviral vectors are comprised of cis-acting long terminal repeats with packaging capacity for up to 6-10 kb of foreign sequence. The minimum cis-acting LTRs are sufficient for replication and packaging of the vectors, which are then used to integrate the therapeutic gene into the target cell to provide permanent transgene expression. Widely used retroviral vectors include those based upon murine leukemia virus (MuLV), gibbon ape leukemia virus (GaLV), Simian Immunodeficiency virus (SIV), human immunodeficiency virus (HIV), and combinations thereof (See, e.g., Buchschacher et al. (1992); Johann et al. (1992); Sommerfelt et al. (1990); Wilson et al. (1989); Miller et al. (1991); PCT International Publication No. WO/1994/026877A1).

At least six viral vector approaches are currently available for gene transfer in clinical trials, which utilize approaches that involve complementation of defective vectors by genes inserted into helper cell lines to generate the transducing agent.

pLASN and MFG-S are examples of retroviral vectors that have been used in clinical trials (See Dunbar et al., 1995; Kohn et al., 1995; Malech et al., 1997). PA317/pLASN was the first therapeutic vector used in a gene therapy trial (Blaese et al., 1995). Transduction efficiencies of 50% or greater have been observed for MFG-S packaged vectors. (Ellem et al., (1997); Dranoff et al., 1997).

Packaging cells are used to form virus particles that are capable of infecting a host cell. Such cells include 293 cells, which package adenovirus, AAV, and Psi-2 cells or PA317 cells, which package retrovirus. Viral vectors used in gene therapy are usually generated by a producer cell line that packages a nucleic acid vector into a viral particle. The vectors typically contain the minimal viral sequences required for packaging and subsequent integration into a host (if applicable), other viral sequences being replaced by an expression cassette encoding the protein to be expressed. The missing viral functions are supplied in trans by the packaging cell line. For example, AAV vectors used in gene therapy typically only possess inverted terminal repeat (ITR) sequences from the AAV genome which are required for packaging and integration into the host genome. Viral DNA is packaged in a cell line, which contains a helper plasmid encoding the other AAV genes, namely rep and cap, but lacking ITR sequences. The cell line is also infected with adenovirus as a helper. The helper virus promotes replication of the AAV vector and expression of AAV genes from the helper plasmid. The helper plasmid is not packaged in significant amounts due to a lack of ITR sequences. Contamination with adenovirus can be reduced by, e.g., heat treatment to which adenovirus is more sensitive than AAV. Additionally, AAV can be produced at clinical scale using baculovirus systems (see U.S. Pat. No. 7,479,554).

In many gene therapy applications, it is desirable that the gene therapy vector be delivered with a high degree of specificity to a particular tissue type. Accordingly, a viral vector can be modified to have specificity for a given cell type by expressing a ligand as a fusion protein with a viral coat protein on the outer surface of the virus. The ligand is chosen to have affinity for a receptor known to be present on the cell type of interest. For example, Han et al. (1995) reported that Moloney murine leukemia virus can be modified to express human heregulin fused to gp70, and the recombinant virus infects certain human breast cancer cells expressing human epidermal growth factor receptor. This principle can be extended to other virus-target cell pairs, in which the target cell expresses a receptor and the virus expresses a fusion protein comprising a ligand for the cell-surface receptor. For example, filamentous phage can be engineered to display antibody fragments (e.g., FAB or Fv) having specific binding affinity for virtually any chosen cellular receptor. Although the above description applies primarily to viral vectors, the same principles can be applied to nonviral vectors. Such vectors can be engineered to contain specific uptake sequences which favor uptake by specific target cells.

Gene therapy vectors can be delivered in vivo by administration to an individual patient, typically by systemic administration (e.g., intravitreal, intravenous, intraperitoneal, intramuscular, subdermal, or intracranial infusion) or topical application, as described below. Alternatively, vectors can be delivered to cells ex vivo, such as cells explanted from an individual patient (e.g., lymphocytes, bone marrow aspirates, tissue biopsy) or universal donor hematopoietic stem cells, followed by reimplantation of the cells into a patient, optionally after selection for cells which have incorporated the vector. A non-limiting exemplary ex vivo approach may involve removal of tissue (e.g., peripheral blood, bone marrow, and spleen) from a patient for culture, nucleic acid transfer to the cultured cells (e.g., hematopoietic stem cells), followed by grafting the cells to a target tissue (e.g., bone marrow, and spleen) of the patient. In some embodiments, the stem cell or hematopoietic stem cell may be further treated with a viability enhancer.

Ex vivo cell transfection for diagnostics, research, or for gene therapy (e.g., via re-infusion of the transfected cells into the host organism) is well known to those of skill in the art. In a preferred embodiment, cells are isolated from the subject organism, transfected with a nucleic acid composition, and re-infused back into the subject organism (e.g., patient). Various cell types suitable for ex vivo transfection are well known to those of skill in the art (See, e.g., Freshney, “Culture of Animal Cells, A Manual of Basic Technique and Specialized Applications (6th edition, 2010) and the references cited therein for a discussion of how to isolate and culture cells from patients).

Suitable cells include, but are not limited to, eukaryotic cells and/or cell lines. Non-limiting examples of such cells or cell lines generated from such cells include COS, CHO (e.g., CHO-S, CHO-K1, CHO-DG44, CHO-DUXB11, CHO-DUKX, CHOK1SV), VERO, MDCK, WI38, V79, B14AF28-G3, BHK, HaK, NSO, SP2/0-Ag14, HeLa, HEK293 (e.g., HEK293-F, HEK293-H, HEK293-T), perC6 cells, any plant cell (differentiated or undifferentiated), as well as insect cells such as Spodoptera fugiperda (Sf), or fungal cells such as Saccharomyces, Pichia and Schizosaccharomyces. In certain embodiments, the cell line is a CHO-K1, MDCK or HEK293 cell line. Additionally, primary cells may be isolated and used ex vivo for reintroduction into the subject to be treated following treatment with a guided nuclease system (e.g. CRISPR/Cas). Suitable primary cells include peripheral blood mononuclear cells (PBMC), and other blood cell subsets such as, but not limited to, CD4+ T cells or CD8+ T cells. Suitable cells also include stem cells such as, by way of example, embryonic stem cells, induced pluripotent stem cells, hematopoietic stem cells (CD34+), neuronal stem cells and mesenchymal stem cells.

In one embodiment, stem cells are used in ex vivo procedures for cell transfection and gene therapy. The advantage to using stem cells is that they can be differentiated into other cell types in vitro, or can be introduced into a mammal (such as the donor of the cells) where they will engraft in the bone marrow. Methods for differentiating CD34+ cells in vitro into clinically important immune cell types using cytokines such a GM-CSF, IFN-gamma, and TNF-alpha are known (as a non-limiting example see, Inaba et al., 1992).

Stem cells are isolated for transduction and differentiation using known methods. For example, stem cells are isolated from bone marrow cells by panning the bone marrow cells with antibodies which bind unwanted cells, such as CD4+ and CD8+ (T cells), CD45+ (panB cells), GR-1 (granulocytes), and Iad (differentiated antigen presenting cells) (as a non-limiting example, see Inaba et al., 1992). Stem cells that have been modified may also be used in some embodiments.

Vectors (e.g., retroviruses, liposomes, etc.) containing therapeutic nucleic acid compositions can also be administered directly to an organism for transduction of cells in vivo. Administration is by any of the routes normally used for introducing a molecule into ultimate contact with blood or tissue cells including, but not limited to, injection, infusion, topical application (e.g., eye drops and cream) and electroporation. Suitable methods of administering such nucleic acids are available and well known to those of skill in the art, and, although more than one route can be used to administer a particular composition, a particular route can often provide a more immediate and more effective reaction than another route. According to some embodiments, the composition is delivered via IV injection.

Vectors suitable for introduction of transgenes into immune cells (e.g., T-cells) include non-integrating lentivirus vectors. See, e.g., U.S. Publication No. 2009/0117617.

As mentioned above, the compositions described herein may be delivered to a target cell using a non-integrating lentiviral particle method, e.g. a LentiFlash® system. Such a method may be used to deliver mRNA or other types of RNAs into the target cell, such that delivery of the RNAs to the target cell results in assembly of the compositions described herein inside of the target cell. See also PCT International Publication Nos. WO/2013/014537, WO/2014/016690, WO/2016/185125, WO/2017/194902, and WO/2017/194903.

Pharmaceutically acceptable carriers are determined in part by the particular composition being administered, as well as by the particular method used to administer the composition. Accordingly, there is a wide variety of suitable formulations of pharmaceutical compositions available, as described below (See, e.g., Remington's Pharmaceutical Sciences, 17th ed., 1989).

The disclosed compositions and methods may also be used in the manufacture of a medicament for treating a disease or disorder in a patient.

### Mechanisms of Action for B2M Safe Harbor Knock-in Methods

Without being bound by any theory or mechanism, the instant invention may be utilized to apply a CRISPR nuclease to process a B2M allele in order to introduce a sequence to a safe harbor site within the B2M allele and thereby control expression of the introduced sequence by the promoter of the B2M allele. A specific guide sequence may be selected from Table 1 based on the targeted position and the type of CRISPR nuclease used (e.g. according to a required PAM sequence).

The B2M gene is located on chromosome 15 and encodes the Beta-2 microglobulin protein. A donor molecule may be used to introduce a desired sequence of nucleotides into a B2M safe harbor site via knock-in.

One strategy is to knock-in a sequence of nucleotides in the first intron or the first intron that follows the first coding exon of the B2M gene. This strategy utilizes an RNA molecule to target a CRISPR nuclease to Intron 1 of the B2M gene and thereby mediate a double-stranded break. Since the break is mediated in a nonregulatory region, it is not expected to affect the expression of the gene. For knock-in of a sequence without a knockout of B2M, the sequence is inserted as a new exon (namely, Exon 2), by adding splicing acceptor (SA) and splicing donor (SD) elements to the knock-in donor cassette. In addition, to prevent the interruption of the expression of the B2M gene, the donor cassette includes a 2A self-cleaving peptide and/or the signal peptide of B2M at the C-terminus of the inserted gene, which is cleaved in the endoplasmic reticulum and enables the separation of the inserted sequence protein expression product from B2M protein.

Another strategy is to knock-in a sequence of nucleotides in the B2M gene by replacing the stop codon. In this case a biallelic break will be mediated in a 3′UTR region either up to 150 nucleotides downstream of the stop codon or upstream to the stop codon but in an intron region. The location of the break in these regions does not affect the expression of B2M. The knocked-in sequence of nucleotides is inserted in place of the stop codon and the knock-in donor cassette contains a 2A self-cleaving peptide at the N-terminus of the inserted sequence of nucleotides enabling the separation of the inserted sequence protein expression product from B2M protein.

Examples of RNA Guide Sequences which Specifically Target Alleles of the B2M Gene

Although a large number of guide sequences can be designed to target a B2M allele, the nucleotide sequences described in Table 1 identified by SEQ ID NOs: 1-28998 below were specifically selected to effectively implement the methods set forth herein.

Table 1 lists guide sequences designed for use as described in the embodiments above to associate specific sequences within a B2M allele. Each engineered guide molecule is further designed such as to associate with a target genomic DNA sequence of interest that lies next to a protospacer adjacent motif (PAM), e.g., a PAM matching the sequence NGG or NAG, where “N” is any nucleobase. The guide sequences were designed to work in conjunction with one or more different CRISPR nucleases, including, but not limited to, e.g. SpCas9WT (PAM SEQ: NGG), SpCas9.VQR.1 (PAM SEQ: NGAN), SpCas9.VQR.2 (PAM SEQ: NGNG), SpCas9.EQR (PAM SEQ: NGAG), SpCas9.VRER (PAM SEQ: NGCG), SaCas9WT (PAM SEQ: NNGRRT), SpRY (PAM SEQ: NRN or NYN), NmCas9WT (PAM SEQ: NNNNGATT), Cpf1 (PAM SEQ: TTTV), JeCas9WT (PAM SEQ: NNNVRYM), OMNI-50 (PAM SEQ: NGG), OMNI-79 (PAM SEQ: NGG), OMNI-103 (PAM SEQ: NNRACT), OMNI-159 (NNNNCMAN), or OMNI-124 (PAM SEQ: NNGNRMNN).

Additional description of OMNI CRISPR nucleases is provided in PCT International Application Publication No. WO 2023/019269 A2, PCT International Application Publication Nos. WO 2022/170199 A2 and WO 2023/107946 A2, U.S. Pat. No. 11,666,641 B2 and PCT International Application Publication Nos. WO 2020/223514 A2, WO 2022/098693 A1, and WO 2023/019263 A1, U.S. Application Publication No. 2023/0122086 A1 and PCT International Application Publication Nos. WO 2021/248016 A2 and WO 2023/102407 A2, PCT International Application Publication No. WO 2022/087135 A1, and PCT International Application Publication No. WO 2022/226215 A1, the contents of each of which are hereby incorporated by reference.

RNA molecules of the present invention are each designed to form complexes in conjunction with one or more different CRISPR nucleases and designed to target polynucleotide sequences of interest utilizing one or more different PAM sequences respective to the CRISPR nuclease utilized.

TABLE 1
Guide sequence portions designed to associate
with specific B2M gene targets

| | SEQ ID | SEQ ID | SEQ ID |
|---|---|---|---|
| | NOs: of 20 | NOs: of 21 | NOs: of 22 |
| Target | base guides | base guides | base guides |
| 15:44716329-44716356 | 1-2 | N/A | N/A |
| Exon 3 | | | |
| 15:44717607-44717813 | 3-310 | 311-574 | 575-852 |
| Exon 4 | | | |
| 15:44717825-44718145 | 853-1286 | 1287-1654 | 1655-2040 |
| Exon 4 | | | |
| 15:44716357-44717323 | 2041-3710 | 3711-5340 | 5341-6996 |
| Intron 3 | | | |
| 15:44717327-44717606 | 6997-7433 | 7434-7817 | 7818-8229 |
| Intron 3 | | | |
| 15:44711614-44712998 | 8230-10585 | 10586-12871 | 12872-15227 |
| Intron 1 | | | |
| 15:44713001-44713065 | 15228-15271 | 15272-15309 | 15310-15352 |
| Intron 1 | | | |
| 15:44713067-44713182 | 15353-15516 | 15517-15676 | 15677-15837 |
| Intron 1 | | | |
| 15:44713184-44713461 | 15838-16135 | 16136-16405 | 16406-16681 |
| Intron 1 | | | |
| 15:44713463-44714419 | 16682-18343 | 18344-19901 | 19902-21501 |
| Intron 1 | | | |
| 15:44714421-44714474 | 21502-21555 | 21556-21603 | 21604-21651 |
| Intron 1 | | | |
| 15:44714476-44715422 | 21652-23029 | 23030-24371 | 24372-25743 |
| Intron 1 | | | |
| 15:44715702-44716328 | 25744-26866 | 26867-27918 | 27919-28998 |
| Intron 2 | | | |

The indicated locations listed in column 1 of Table 1 are based on gnomAD v3 database and UCSC Genome Browser assembly ID: hg38, Sequencing/Assembly provider ID: Genome Reference Consortium Human GRCh38.p12 (GCA_000001405.27). Assembly date: December 2013 initial release; December 2017 patch release 12.

Examples are provided below to facilitate a more complete understanding of the invention. The following examples illustrate the exemplary modes of making and practicing the invention. However, the scope of the invention is not limited to specific embodiments disclosed in these Examples, which are for purposes of illustration only.

### EXPERIMENTAL DETAILS

### Example 1: B2M On-Target Activity Analysis

Guide sequences comprising 17-50 contiguous nucleotides containing nucleotides in the sequence set forth in any one of SEQ ID NOs: 1-28998 are screened for high on target activity using a CRISPR nuclease in a target cell. On-target activity is determined by DNA capillary electrophoresis analysis.

### Example 2: Insertion of a Sequence of Interest at a B2M Safe Harbor Site

B2M is highly expressed in target cells (e.g. macrophages) and contains a signal peptide (SP) in its first exon. These properties make a B2M locus a great candidate for integration and secretion of any gene of interest. To take advantage of the B2M SP, a strategy was designed to insert a donor DNA in the first intron of B2M downstream to the SP. This homology directed repair (HDR) dependent insertion is enabled by CRISPR-induced DNA damage paired with a donor sequence encoding a gene of interest. The strategy aims to enable both expression and secretion of the gene of interest while maintaining the expression and function of the safe harbor site-containing gene.

### CRISPR-Based Ribonucleoprotein (RNP) Composition Allows Editing in Intron 1 of B2M

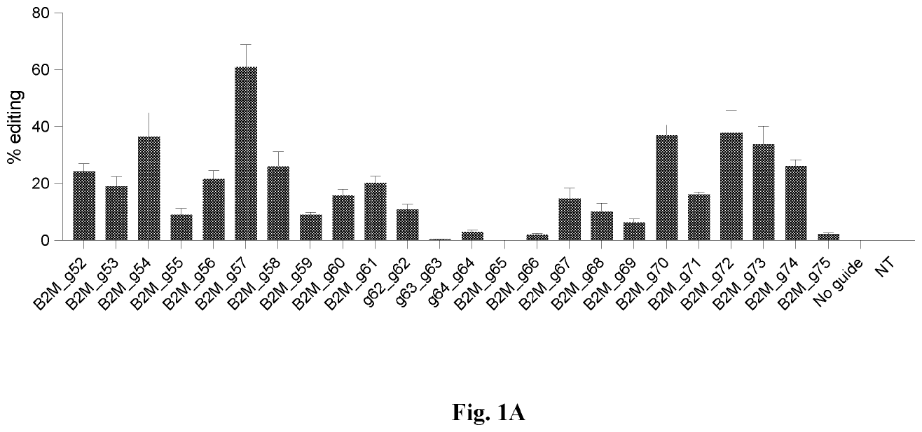

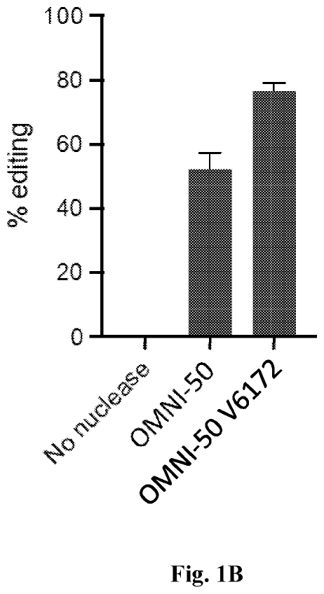

A guide screen in HeLa cells was performed to identify a potential CRISPR-based RNP composition which allows for editing in Intron 1 of B2M. Briefly, an OMNI-50 nuclease coding plasmid (64 ng) was co-transfected with each of the guide expressing plasmids (20 ng) in a 96-well plate format using jetOPTIMUS reagent (Polyplus). Cells were harvested 72 hours post-transfection, genomic DNA was extracted, and the DNA was analyzed using next generation sequencing (NGS). The observed percent editing (%) as measured by indel abundance varied between the guides used and resulted in up to 61% editing using the OMNI-50 nuclease (FIG. 1A).

The top performing guide was then examined in hematopoietic stem cells (HSCs) using electroporation of RNPs. A composition of a single-guide RNA (sgRNA) (120 pmole) with either an OMNI-50 or OMNI-50 V6172 nuclease (105 pmole) was electroporated into cells using Lonza P3 cell 4D-nucleofecuor X Kiu S (CA-137 program, PBC2-00675, Lonza). 72 hours later HSCs were harvested and their genomic DNA was analyzed using NGS. The analysis revealed highly efficient editing levels of nearly 80% with the OMNI-50 V6172 nuclease and lower efficiency with the OMNI-50 nuclease (FIG. 1B). As a result, the current composition chosen for editing and performing HDR in Intron 1 of B2M is a B2M_g57 guide molecule+OMNI-50 V6172 nuclease.

### HDR Editing Template Integration in Intron 1 of B2M

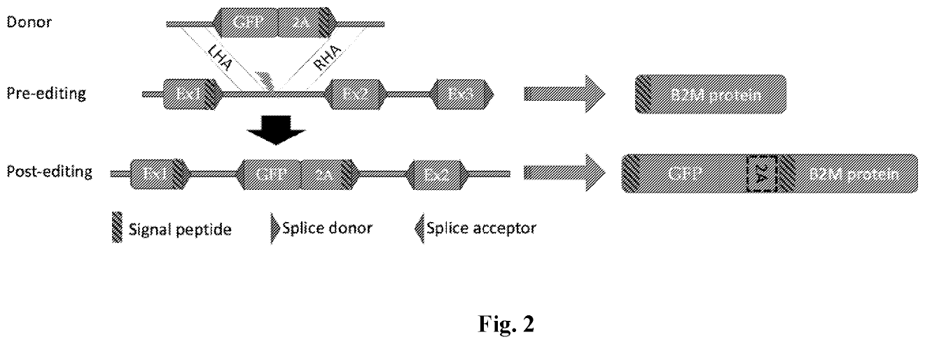

AAV particles carrying a donor construct encoding for GFP fused to the self-cleaving P2A-T2A elements (2A) and SP were generated to examine the feasibility of HDR editing template integration in Intron 1 of B2M. As shown in FIG. 2, the donor targets Intron 1 of B2M downstream to the B2M SP. The donor construct contains a GFP-2A-SP sequence flanked by a splice acceptor and splice donor, and further flanked by 800 basepair homology arms matching the B2M_g57 cleavage site (see SEQ ID NO: 29012 and Table 4). CRISPR-mediated DNA cleavage facilitates HDR integration of the donor sequence into B2M Intron 1. This results in a bicistronic transcript with a single open reading frame encoding for SP-GFP-2A-SP-B2M which, following 2A self-cleaving, results in two separated polypeptides of SP-GFP and SP-B2M. Importantly, since the donor does not contain any promoter or other mRNA-stabilizing elements, GFP cannot be expressed without successful integration into the desired locus.

GFP Integration into a B2M Locus in HSCs

The above approach was first examined in primary HSCs. Cells were electroporated with an RNP composed of an OMNI-50 V6172 nuclease and a B2M_g57 guide molecule, followed by infection with AAV6 particles carrying a GFP donor molecule at a multiplicity of infection (MOI) of 10⁵.

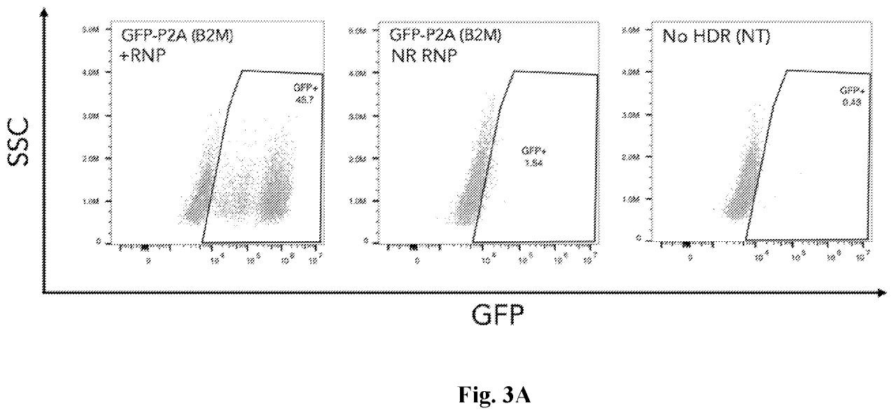

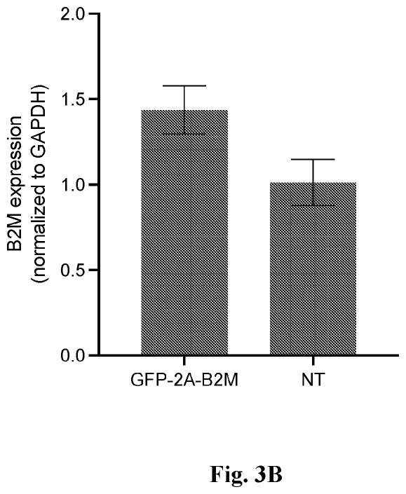

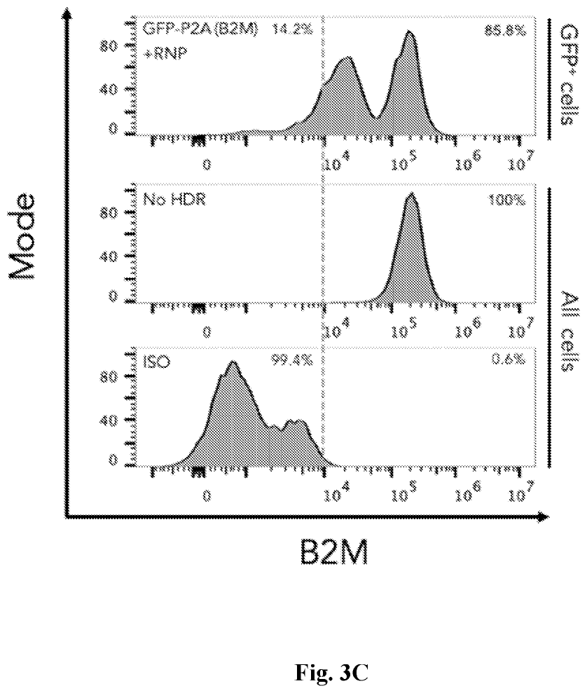

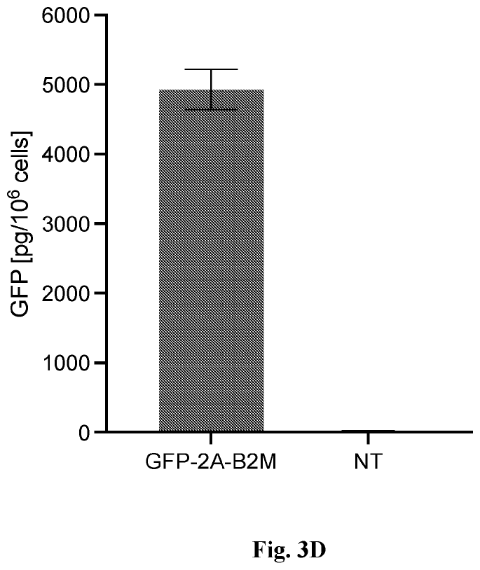

First, HSCs were analyzed by FACS for GFP expression three days following treatment (FIG. 3A). A combination of the RNP (B2M_g57 guide) with the donor AAV resulted in 46% GFP-expressing cells (average of two repeats).

To confirm that GFP expression was dependent on successful integration into a B2M locus, we examined GFP expression in a group infected with the donor AAV combined with irrelevant RNP (NR RNP). No significant GFP expression was observed in this group as it matched the untreated HSCs control group. To confirm the expression of B2M is maintained after HDR integration, we then examined B2M transcript and protein levels at the Day 7 timepoint.

To confirm that GFP integration into a B2M locus maintains the transcription of the safe harbor gene, the total B2M transcript level was assessed using qRT-PCR with a primer set which targets Exon 2 of B2M, which is downstream to the editing site and thus identifies both the native and the GFP-integrated transcripts (FIG. 3B). The transcript levels of the HDR group showed slightly higher levels compared to those of the untreated control group, thus no reduction in B2M transcripts levels was detected.

To assess the protein levels, we analyzed the cells by FACS (FIG. 3C). To better understand the effect of HDR on B2M expression, the analysis focused on GFP⁺ cells in the GFP-P2A-B2M+RNP group compared to “No HDR” and “ISO-staining” (non-targeting but labeled antibody set) groups. Based on the last group, background from staining was determined (i.e. no B2M expression). While the majority of GFP⁺ cells (~86%) express B2M protein, we detected a second, lower intensity peak which retained intermediate B2M expression representing ~37% of the GFP⁺ cells. This population might be explained by attenuated translation of the gene located downstream to the 2A peptides. We also observed ~14% of the cells having signal comparable to the ISO-staining group, which indicates very low to no-expression.

Finally, to examine whether GFP is successfully secreted from HSCs following integration into B2M locus, the cell media was sampled using GFP ELISA kit (Abcam, ab171581). HSCs in the HDR group secreted ~1.3 ng GFP/10⁶ cells while no GFP was detected in the control group (FIG. 3D—note that this value is the absolute GFP level detected after considering the media volume).

Differentiation of Edited HSCs into Macrophages Maintains GFP Expression from a B2M Locus

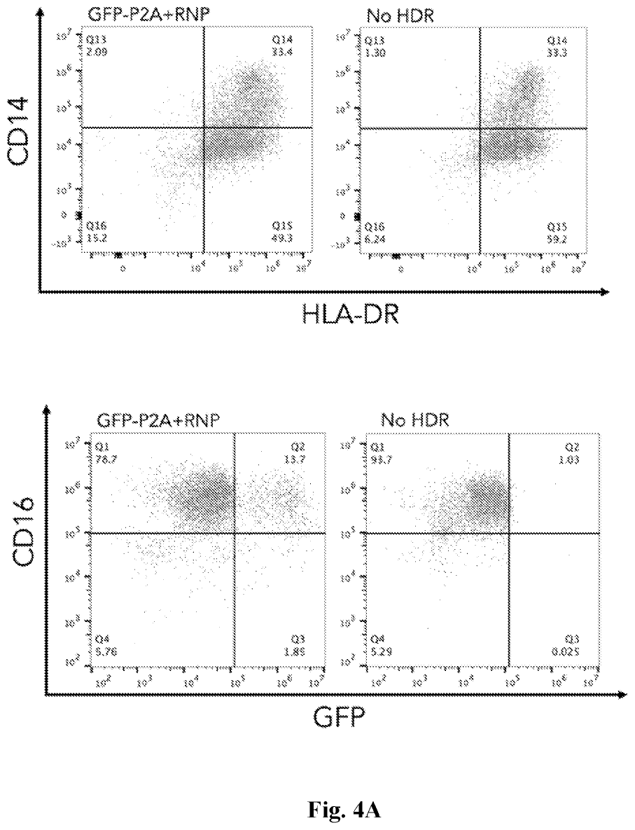

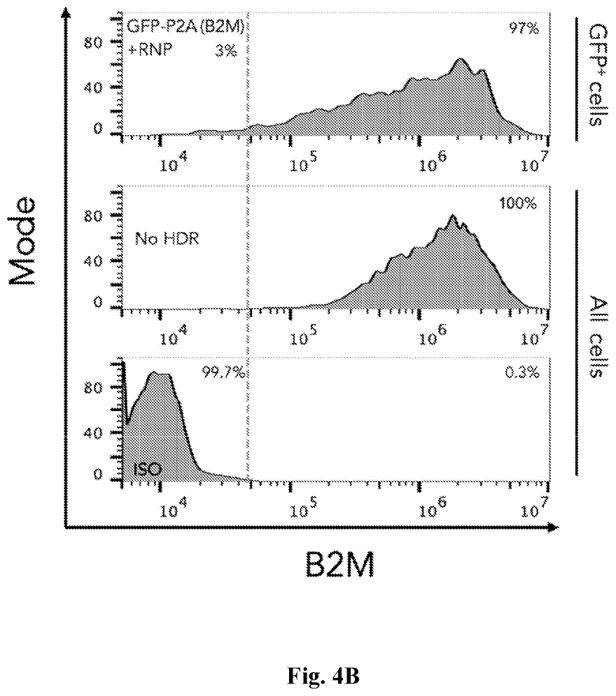

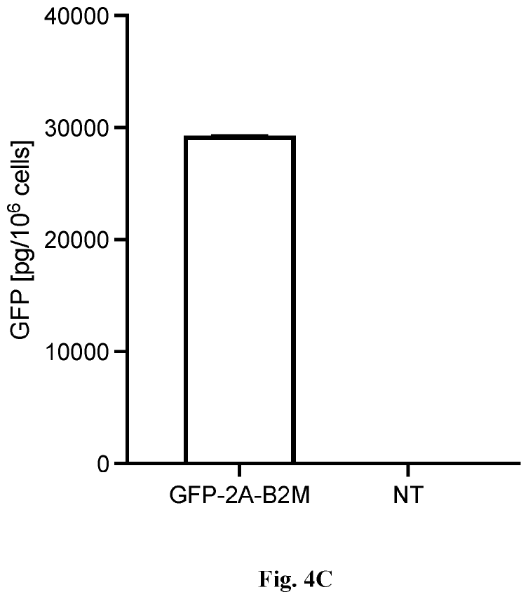

Edited HSCs can be differentiated into numerous hematopoietic cells lineages with various therapeutic potential. Here we examine whether edited HSCs retain GFP expression and secretion after differentiation into macrophages. To that end, edited HSCs were differentiated into macrophages as previously described (Gomez-Ospina et al, Nature Communications, 2019). In short, cells were plated in differentiation media (SFEM II supplemented with SCF (200 ng/ml), Il-3 (10 ng/ml), IL-6 (10 ng/ml), FLT3-L (50 ng/ml), M-CSF (10 ng/ml), GM-CSF (10 ng/ml), penicillin/streptomycin (10 U/mL) for 48 hours. Adherent cells were maintained in maintenance medium (RPMI supplemented with FBS (10% v/v), M-CSF (10 ng/ml), GM-CSF (10 ng/ml), and penicillin/streptomycin (10 U/mL)), for 19 days. To validate the differentiation procedure, cells were scraped and analyzed using the following FACS panel: CD14, CD16, and HLA-DR. As shown in FIG. 4A, by inspection of the staining panels of edited and un-edited controls it can be seen that most of the cells examined were successfully differentiated into macrophages and expressed all tested macrophage markers. The percentage of macrophages in the population is unaltered by editing and was comparable between edited and un-edited controls. Furthermore, looking at the FITC channel in the FACS analysis, we can see that ~20% of the cells expressed GFP even after differentiation.

B2M expression signal was also measured using FACS to confirm that the B2M gene is still expressed after the differentiation procedure and HDR (FIG. 4B). While we detected a reduction in B2M expression at the Day 7 timepoint in the originating HSCs, here we detected recovery of B2M expression as almost all GFP-expressing cells were B2M+ and over 90% exhibited WT-levels of signal. To examine GFP secretion, we performed an ELISA assay of the cell media (FIG. 4C) and observed GFP secretion in the edited population. This confirms that GFP insertion into a B2M locus allows both expression and secretion of a gene of interest.

GFP Integration into a B2M Locus in iPSCs

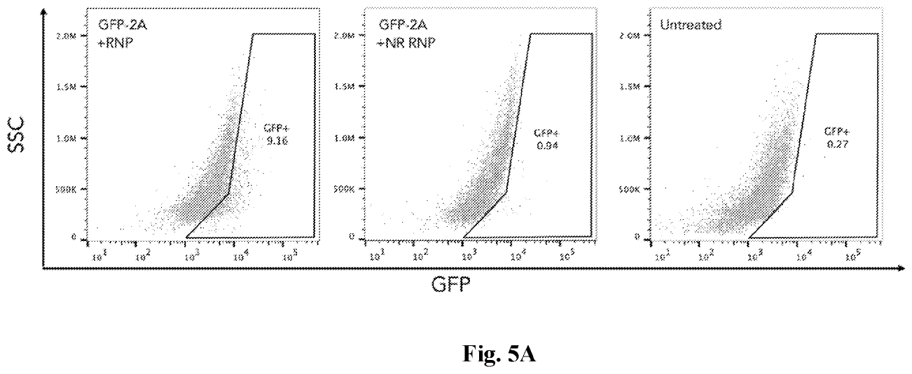

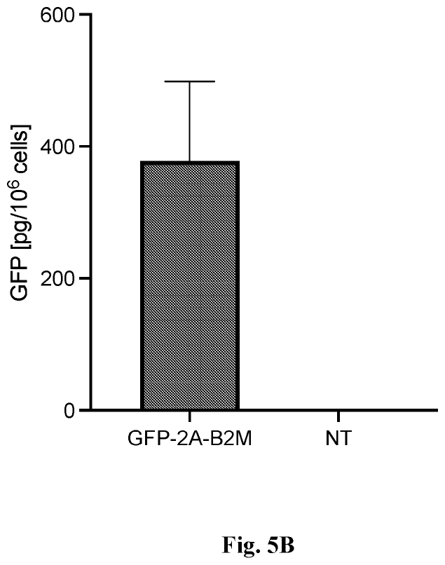

To confirm that this system is relevant in other multipotent cell types, we repeated these experiments in iPSCs. Similar to HSCs, we delivered RNP using electroporation and delivered the donor DNA molecule using AAV. For a high infection rate of iPSCs, we used AAV-DJ. Examination of GFP expression in iPSCs three days post-treatment showed GFP is successfully expressed in iPSCs after HDR integration (FIG. 5A). Echoing results shown with HSCs, integration of a GFP cassette is essential for GFP expression as control groups without specific CRISPR activity did not exhibit GFP expression. Secretion of GFP from modified iPSCs was assessed by ELISA (FIG. 5B). GFP secretion was detected only in HDR groups, confirming that GFP integration into Intron 1 of B2M enables expression of a polypeptide containing the SP which leads to its subsequent secretion.

FACS Analysis in iPSCs

TABLE 2
Guide sequence portions that are 22-nucleotides in
length and target R2M intron 1

| Guide Sequence | |
|---|---|
| Portion Name | Guide Sequence Portion |
| g52 | GUCCCAGGAAUCACUUGAACCC |
| | (SEQ ID NO: 25307) |
| g53 | UUCAAGUGAUUCCUGGGACUAC |
| | (SEQ ID NO: 25648) |
| g54 | CUCCUGGGUUCAAGUGAUUCCU |
| | (SEQ ID NO: 24977) |
| g55 | CCUCCUGGGUUCAAGUGAUUCC |
| | (SEQ ID NO: 24916) |
| g56 | UGAAGUGUCUAGUAGAGUGCCU |
| | (SEQ ID NO: 25521) |
| g57 | AUGAAGUGUCUAGUAGAGUGCC |
| | (SEQ ID NO: 24696) |
| g58 | AAAAUCAGAUGGGUGUAGAUCA |
| | (SEQ ID NO: 24384) |
| g59 | UCAGAUGGGUGUAGAUCAAGGC |
| | (SEQ ID NO: 25443) |
| g60 | GGGUGUAGAUCAAGGCAGGAGC |
| | (SEQ ID NO: 25268) |
| g61 | CAGGAGCAGGAACCAAAAAGAA |
| | (SEQ ID NO: 24820) |
| g62 | AUGAGGGAAAGAUACCAAGUCA |
| | (SEQ ID NO: 24700) |
| g63 | AGUCACGGUUUAUUCUUCAAAA |
| | (SEQ ID NO: 24629) |
| g64 | CACGGUUUAUUCUUCAAAAUGG |
| | (SEQ ID NO: 24794) |
| g65 | UUUUGAAGAAUAAACCGUGACU |
| | (SEQ ID NO: 25735) |
| g66 | GGUUUAUUCUUCAAAAUGGAGG |
| | (SEQ ID NO: 25287) |
| g67 | CUUCAAAAUGGAGGUGGCUUGU |
| | (SEQ ID NO: 25019) |
| g68 | UUCAAAAUGGAGGUGGCUUGUU |
| | (SEQ ID NO: 25646) |
| g69 | AAAUGGAGGUGGCUUGUUGGGA |
| | (SEQ ID NO: 24410) |
| g70 | UGGAGGUGGCUUGUUGGGAAGG |
| | (SEQ ID NO: 25578) |
| g71 | GUUGGGAAGGUGGAAGCUCAUU |
| | (SEQ ID NO: 25349) |
| g72 | GUGGAAGCUCAUUUGGCCAGAG |
| | (SEQ ID NO: 25326) |
| g73 | GCUCAUUUGGCCAGAGUGGAAA |
| | (SEQ ID NO: 25189) |
| g74 | UUGGCCAGAGUGGAAAUGGAAU |
| | (SEQ ID NO: 25688) |
| g75 | UGGCCAGAGUGGAAAUGGAAUU |
| | (SEQ ID NO: 25584) |

TABLE 3
Details of CRISPR nucleases used in Example 2 and
a compatible sgRNA scaffold sequence. Additional
descriptions of the OMNI-50 CRISPR nuclease and
its variants are provided in U.S. Pat. No.
11,666,641 and PCT International Application
Publication Nos. WO 2022/098693 A1 and WO 2023/
019263A1, the contents of each of which are hereby
incorporated by reference.

| CRISPR | PAM | |
|---|---|---|
| Nuclease | Sequence | sgRNA Scaffold Sequence |
| OMNI-50 | NGG | GUUUGAGAGUUAUGAAAAUGACGAGUU |
| (SEQ ID NO: | | CAAAUAAAAAUUUAUUCAAACCGCCUA |
| 29004) | | UUUAUAGGCCGCAGAUGUUCUGCUUU |
| | | (SEQ ID NO: 29006) |
| OMNI-50 | NGG | GUUUGAGAGUUAUGAAAAUGACGAGUU |
| V6172 | | CAAAUAAAAAUUUAUUCAAACCGCCUA |
| (SEQ ID NO: | | UUUAUAGGCCGCAGAUGUUCUGCUUU |
| 29005) | | (SEQ ID NO: 29006) |

TABLE 4
Sequence elements encoded by the RHA(800 bp)-GFP-P2A-B2M-LHA(800 bp) HDR
template molecule described in Example 2, above (SEQ ID NO: 29012).

| Element | Sequence |
|---|---|
| Splice acceptor | TACTGACATCCACTTTGCCTTTCTCTCCACAG (SEQ ID NO: 29007) |
| sequence | |
| GFP | GTGAGCAAGGGCGAGGAGCTGTTCACCGGGGTGGTGCCCATCCT |
| | GGTCGAGCTGGACGGCGACGTAAACGGCCACAAGTTCAGCGTGT |
| | CCGGCGAGGGCGAGGGCGATGCCACCTACGGCAAGCTGACCCTG |
| | AAGTTCATCTGCACCACCGGCAAGCTGCCCGTGCCCTGGCCCACC |
| | CTCGTGACCACCCTGACCTACGGCGTGCAGTGCTTCAGCCGCTAC |
| | CCCGACCACATGAAGCAGCACGACTTCTTCAAGTCCGCCATGCC |
| | CGAAGGCTACGTCCAGGAGCGCACCATCTTCTTCAAGGACGACG |
| | GCAACTACAAGACCCGCGCCGAGGTGAAGTTCGAGGGCGACACC |
| | CTGGTGAACCGCATCGAGCTGAAGGGCATCGACTTCAAGGAGGA |
| | CGGCAACATCCTGGGGCACAAGCTGGAGTACAACTACAACAGCC |
| | ACAACGTCTATATCATGGCCGACAAGCAGAAGAACGGCATCAAG |
| | GTGAACTTCAAGATCCGCCACAACATCGAGGACGGCAGCGTGCA |
| | GCTCGCCGACCACTACCAGCAGAACACCCCCATCGGCGACGGCC |
| | CCGTGCTGCTGCCCGACAACCACTACCTGAGCACCCAGTCCGCCC |
| | TGAGCAAAGACCCCAACGAGAAGCGCGATCACATGGTCCTGCTG |
| | GAGTTCGTGACCGCCGCCGGGATCACTCTCGGCATGGACGAGCT |
| | GTACAAG (SEQ ID NO: 29008) |
| HA-P2A-T2A | TCCTACCCATACGATGTTCCAGATTACGCGGCCGCTGCCACCAAC |
| | TTCAGCCTGCTGAAGCAGGCCGGCGACGTGGAGGAGAACCCCGG |
| | CCCCGGAAGCGGAGAGGGCAGAGGAAGTCTGCTAACATGCGGT |
| | GACGTCGAGGAGAATCCTGGACCT (SEQ ID NO: 29009) |
| APOH-signal | ATGATTTCTCCAGTGCTCATCTTGTTCTCGAGTTTTCTCTGCCATG |
| peptide | TTGCTATTGCAGGACGG (SEQ ID NO: 29010) |
| Splice donor | GTAAGTTCAAGGTTACAAGACAGGTTTAAGGAGACCAATAGAAA |
| sequence | CTG (SEQ ID NO: 29011) |

### REFERENCES

1. Ahmad and Allen (1992) “Antibody-mediated Specific Binging and Cytotoxicity of Lipsome-entrapped Doxorubicin to Lung Cancer Cells in Vitro”, Cancer Research 52:4817-20.
2. Anderson (1992) “Human gene therapy”, Science 256:808-13.
3. Anzalone et al. (2019) “Search-and-replace genome editing without double-strand brakes or donor DNA”, Nature 576, 149-157.
4. Basha et al. (2011) “Influence of Cationic Lipid Composition on Gene Silencing Properties of Lipid Nanoparticle Formulations of siRNA in Antigen-Presenting Cells”, Mol. Ther. 19 (12): 2186-200.
5. Behr (1994) Gene transfer with synthetic cationic amphiphiles: Prospects for gene therapy”, Bioconjuage Chem 5:382-89.
6. Blaese (1995) “Vectors in cancer therapy: how will they deliver”, Cancer Gene Ther. 2:291-97.
7. Blaese et al. (1995) “T lympocyte-directed gene therapy for ADA-SCID: initial trial results after 4 years”, Science 270 (5235): 475-80.
8. Buchschacher and Panganiban (1992) “Human immunodeficiency virus vectors for inducible expression of foreign genes”, J. Virol. 66:2731-39.
9. Burstein et al. (2017) “New CRISPR-Cas systems from uncultivated microbes”, Nature 542:237-41
10. Chang and Wilson (1987) “Modification of DNA ends can decrease end-joining relative to homologous recombination in mammalian cells”, Proc. Natl. Acad. Sci. USA 84:4959-4963.
11. Chung et al. (2006) “Agrobacterium is not alone: gene transfer to plants by viruses and other bacteria”, Trends Plant Sci. 11 (1): 1-4.
12. Coelho et al. (2013) “Safety and efficacy of RNAi therapy for transthyretin amyloidosis” N. Engl. J. Med. 369, 819-829.
13. Crystal (1995) “Transfer of genes to humans: early lessons and obstacles to success”, Science 270 (5235): 404-10.
14. Dillon (1993) “Regulation gene expression in gene therapy” Trends in Biotechnology 11 (5): 167-173.
15. Dranoff et al. (1997) “A phase I study of vaccination with autologous, irradiated melanoma cells engineered to secrete human granulocyte macrophage colony stimulating factor”, Hum. Gene Ther. 8 (1): 111-23.
16. Dunbar et al. (1995) “Retrovirally marked CD34-enriched peripheral blood and bone marrow cells contribute to long-term engraftment after autologous transplantation”, Blood 85:3048-57.
17. Ellem et al. (1997) “A case report: immune responses and clinical course of the first human use of granulocyte/macrophage-colony-stimulating-factor-transduced autologous melanoma cells for immunotherapy”, Cancer Immunol Immunother 44:10-20.
18. Gao and Huang (1995) “Cationic liposome-mediated gene transfer” Gene Ther. 2 (10): 710-22.
19. Haddada et al. (1995) “Gene Therapy Using Adenovirus Vectors”, in: The Molecular Repertoire of Adenoviruses III: Biology and Pathogenesis, ed. Doerfler and Böhm, pp. 297-306.
20. Han et al. (1995) “Ligand-directed retro-viral targeting of human breast cancer cells”, Proc Natl Acad Sci U.S.A. 92 (21): 9747-51.
21. Inaba et al. (1992) “Generation of large numbers of dendritic cells from mouse bone marrow cultures supplemented with granulocyte/macrophage colony-stimulating factor”, J Exp Med. 176 (6): 1693-702.
22. Jinek et al. (2012) “A programmable dual-RNA-guided DNA endonuclease in adaptive bacterial immunity,” Science 337 (6096): 816-21.
23. Johan et al. (1992) “GLVR1, a receptor for gibbon ape leukemia virus, is homologous to a phosphate permease of Neurospora crassa and is expressed at high levels in the brain and thymus”, J Virol 66 (3): 1635-40.
24. Judge et al. (2006) “Design of noninflammatory synthetic siRNA mediating potent gene silencing in vivo”, Mol Ther. 13 (3): 494-505.
25. Kohn et al. (1995) “Engraftment of gene-modified umbilical cord blood cells in neonates with adenosine deaminase deficiency”, Nature Medicine 1:1017-23.
26. Kremer and Perricaudet (1995) “Adenovirus and adeno-associated virus mediated gene transfer”, Br. Med. Bull. 51 (1): 31-44.
27. Macdiarmid et al. (2009) “Sequential treatment of drug-resistant tumors with targeted minicells containing siRNA or a cytotoxic drug”, Nat Biotechnol. 27 (7): 643-51.
28. Malech et al. (1997) “Prolonged production of NADPH oxidase-corrected granulocytes after gene therapy of chronic granulomatous disease”, PNAS 94 (22): 12133-38.
29. Miller et al. (1991) “Construction and properties of retrovirus packaging cells based on gibbon ape leukemia virus”, J Virol. 65 (5): 2220-24.
30. Miller (1992) “Human gene therapy comes of age”, Nature 357:455-60.
31. Mitani and Caskey (1993) “Delivering therapeutic genes-matching approach and application”, Trends in Biotechnology 11 (5): 162-66.
32. Nabel and Felgner (1993) “Direct gene transfer for immunotherapy and immunization”, Trends in Biotechnology 11 (5): 211-15.
33. Remy et al. (1994) “Gene Transfer with a Series of Lipophilic DNA-Binding Molecules”, Bioconjugate Chem. 5 (6): 647-54.
34. Sentmanat et al. (2018) “A Survey of Validation Strategies for CRISPR-Cas9 Editing”, Scientific Reports 8:888, doi: 10.1038/s41598-018-19441-8.
35. Sommerfelt et al. (1990) “Localization of the receptor gene for type D simian retroviruses on human chromosome 19”, J. Virol. 64 (12): 6214-20.
36. Van Brunt (1988) “Molecular framing: transgenic animals as bioactors” Biotechnology 6:1149-54.
37. Vigne et al. (1995) “Third-generation adenovectors for gene therapy”, Restorative Neurology and Neuroscience 8 (1,2): 35-36.
38. Weissman and Kariko (2015) “mRNA: Fulfilling the promise of gene therapy”, Molecular Therapy (9): 1416-7.
39. Wilson et al. (1989) “Formation of infectious hybrid virion with gibbon ape leukemia virus and human T-cell leukemia virus retroviral envelope glycoproteins and the gag and pol proteins of Moloney murine leukemia virus”, J. Virol. 63:2374-78.
40. Yu et al. (1994) “Progress towards gene therapy for HIV infection”, Gene Ther. 1 (1): 13-26.
41. Zetsche et al. (2015) “Cpf1 is a single RNA-guided endonuclease of a class 2 CRISPR-Cas system” Cell 163 (3): 759-71.
42. Zuris et al. (2015) “Cationic lipid-mediated delivery of proteins enables efficient protein based genome editing in vitro and in vivo” Nat Biotechnol. 33 (1): 73-80.

1. A method for modifying in a cell at least one allele of the Beta-2 microglobulin (B2M) gene, the method comprising
introducing to the cell a composition comprising:
at least one CRISPR nuclease, or a polynucleotide molecule encoding the CRISPR nuclease; and
an RNA molecule comprising a guide sequence portion having 17-50 nucleotides, or a nucleotide sequence encoding the same,
wherein a complex of the CRISPR nuclease and the RNA molecule affects a double strand break in at least one allele of the B2M gene.

2. The method of claim 1, wherein the composition further comprises a donor molecule comprising a sequence of nucleotides that is introduced at the double strand break site.

3. The method of claim 2, wherein the introduced sequence comprises a sequence from an Alpha-1 antitrypsin, Glucose-6-phosphatase (G6PC), Serpin Family A Member (SERPINA), Transthyretin (TTR), ornithine transcarbamylase, argininosuccinic acid synthetase, arginase, argininosuccinase, carbamoyl phosphate synthetase, and N-acetylglutamate synthetase, Alpha Galactosidase A, Coagulation Factor IX, Coagulation Factor VII, Lysosomal Alpha Glucosidase, Fibrinogen, Phenylalanine 4 Hydroxylase, Alkaline Phosphatase, Glucosylceramidase, Beta Galactosidase, Porphobilinogen Deaminase, Arylsulfatase B, Beta Glucuronidase, Alpha-N-Acetylglucosaminidase, Lysosomal Alpha, Alpha L-Iduronidase, Mannosidase, Phosphatidylcholine Sterol Acyltransferase, N-Sulphoglucosamine Sulphohydrolase, Coagulation Factor X, N-Acetylgalactosamine-6-Sulfatase, Sphingomyelin Phosphodiesterase, iduronate-2-sulfatase, Lysosomal Alpha Glucosidase, Cyclin Dependent Kinase Like 5, Prolow Density Lipoprotein Receptor Related Protein 1, Phenylalanine Ammonia Lyase, Protein Glutamine Gamma Glutamyltransferase K, or Lysosomal Protective Protein encoding gene.

4. The method of claim 2, wherein the introduced sequence comprises a sequence from an acid α-glucosidase, α-L-iduronidase, α-galactosidase, iduronate-2-sulfatase, N-acetylgalactosamine-6-sulfatase, N-acetylgalactosamine-4-sulfatase, a lysophosphatidylcholine metabolism-related protein, preferably phospholipase A2, a T-REC or K-REC related protein, β-glucosidase, β-glucocerebrosidase, arylsulfatase A, Factor VIII, insulin-like growth factor 1 (IGF-1), surfactant protein A, surfactant protein B, aspartyl-β-glucosaminidase, acetyl-CoA α-glucosaminide, acetyl-CoA-arylamine N-acetyltransferase, N-acetylglucosamine-6-sulfatase, N-acetylglucosamine-1-Phosphotransferase, α-N-acetylglucosaminidase, acid ceramidase, aspartacylase, lysosomal acid lipase, acid sphingomyelinase, arylsulfatase B, α-L-fucosidase, galactosylceramidase, galactocerebrosidase, β-galactosidase, protective protein/cathepsin A, β-glucoronidase, heparan N-sulfatase, β-hexosaminidase A, hyaluronidase-1, alpha-D-mannosidase, beta-mannosidase, alpha-neuraminidase, beta-hexosaminidase A, beta-hexosaminidase B, palmitoyl-protein thioesterase, tripeptidyl peptidase I, Battenin, Ceroid-lipofuscinosis neuronal protein 5 (CLN5), Ceroid-lipofuscinosis neuronal protein 6 (CLN6), Ceroid-lipofuscinosis neuronal protein 7 (CLN7), Ceroid-lipofuscinosis neuronal protein 8 (CLN8), (Cathepsin D), cystinosin, cathepsin K, Sialin, Lysosome-associated membrane protein 2 (LAMP2), human growth hormone, follicle-stimulating hormone, erythropoietin, CD-19, a cytokine, a chemokine, IL-10, IGF1, TGF-β, IL-15, CXCR4, IL-4, or a granulocyte colony-stimulating factor (G-CSF) encoding gene.

5. The method of any one of claims 2-4, wherein the donor molecule contains a sequence from a gene encoding a protein that is secreted by a cell.

6. The method of any one of claims 2-5, wherein the introduced sequence comprises a sequence that encodes a polypeptide of interest that is expressed by the cell.

7. The method of claim 6, wherein the expressed polypeptide of interest is secreted by the cell.

8. The method of claim 6 or 7, wherein the expressed polypeptide of interest further comprises a signal peptide.

9. The method of claim 8, wherein the signal peptide is encoded by the allele of the B2M gene.

10. The method of any one of claims 2-9, wherein the introduced sequence comprises a sequence encoding a 2A self-cleaving peptide.

11. The method of any one of claims 2-10, wherein the introduced sequence comprises a sequence that encodes a signal peptide.

12. The method of claim 11, wherein the signal peptide is a B2M signal peptide.

13. The method of any one of claims 2-12, wherein the introduced sequence comprises a splice acceptor sequence and a splice donor sequence.

14. The method of any one of claims 2-13, wherein the introduced sequence comprises a splice acceptor sequence, a sequence encoding a polypeptide of interest, a sequence encoding a 2A self-cleaving peptide, a signal peptide, and a splice donor sequence.

15. The method of any one of claims 2-14, wherein the donor molecule comprises a first homology arm sequence that shares at least 90%, preferably 100%, sequence identity with a B2M sequence upstream of the double-strand break and a second homology arm sequence that shares at least 90%, preferably 100%, sequence identity with a B2M sequence downstream of the double-strand break.

16. The method of claim 15, wherein the first homology arm sequence and second homology arm sequences are each about 20-50, 50-100, 100-200, 200-500, 500-1000, or 1000-2000 nucleotides in length.

17. The method of any one of claims 6-16, wherein the polypeptide of interest is a soluble protein.

18. The method of any one of claims 6-17, wherein the polypeptide of interest is about 20-50, 50-100, 100-200, 200-500, 500-1000, or 1000-2000 amino acids in length.

19. The method of any one of claims 1-18, wherein the RNA molecule comprises a guide sequence portion having 17-50 contiguous nucleotides containing nucleotides in the sequence set forth in any one of SEQ ID NOs: 1-28998.

20. The method of any one of claims 1-19, wherein the RNA molecule comprises a non-discriminatory guide portion that targets Exon 3, Exon 4, Intron 1, Intron 2, Intron 3, or a 3′ untranslated region (3′ UTR) of the B2M gene.

21. The method of any one of claims 1-20, wherein the RNA molecule comprises a non-discriminatory guide portion that targets a sequence that is located within a genomic range selected from any one of 15:44716329-44716356, 15:44717607-44717813, 15:44717825-44718145, 15:44716357-44717323, 15:44717327-44717606, 15:44711614-44712998, 15:44713001-44713065, 15:44713067-44713182, 15:44713184-44713461, 15:44713463-44714419, 15:44714421-44714474, 15:44714476-44715422, and 15:44715702-44716328.

22. The method of any one of claims 1-21, wherein the modified allele of the B2M gene expresses a B2M gene product.

23. The method of any one of claims 6-22, wherein the modified allele of the B2M gene expresses a B2M polypeptide and the polypeptide of interest.

24. The method of any one of claims 1-23, wherein the cell is a stem cell, a monocyte, a macrophage, an iPS-derived monocyte, an iPS-derived macrophage, a hematopoietic stem cell (HSC), a hematopoietic stem and progenitor cell (HSPC), a myeloid precursor cell, a myeloblast, a lymphoblast, an erythroid precursor cell, a platelet cell, a natural killer (NK) cell, a β-lymphocyte, a T-lymphocyte, an eosinophil, a neutrophil, an iPS-derived cell, or a basophil.

25. The method of any one of claims 1-24, wherein the cell is a stem cell, and the method further comprises differentiating the stem cell after modifying the stem cell.

26. A modified cell obtained by the method of any one of claims 1-25.

27. The modified cell of claim 26, wherein the modified cell is a stem cell, a monocyte, a macrophage, an iPS-derived monocyte, an iPS-derived macrophage, a hematopoietic stem cell (HSC), a hematopoietic stem and progenitor cell (HSPC), a myeloid precursor cell, a myeloblast, a lymphoblast, an erythroid precursor cell, a platelet cell, a natural killer (NK) cell, a β-lymphocyte, a T-lymphocyte, an eosinophil, a neutrophil, an iPS-derived cell, or a basophil.

28. A composition comprising an RNA molecule comprising a guide sequence portion having 17-50 contiguous nucleotides containing nucleotides in the sequence set forth in any one of SEQ ID NOs: 1-28998.

29. The composition of claim 28, further comprising at least one CRISPR nuclease.

30. The composition of claim 28 or 29, further comprising a donor molecule.

31. The composition of claim 30, wherein the donor molecule comprises a sequence from an Alpha-1 antitrypsin, Glucose-6-phosphatase (G6PC), Serpin Family A Member (SERPINA), Transthyretin (TTR), ornithine transcarbamylase, argininosuccinic acid synthetase, arginase, argininosuccinase, carbamoyl phosphate synthetase, and N-acetylglutamate synthetase, Alpha Galactosidase A, Coagulation Factor IX, Coagulation Factor VII, Lysosomal Alpha Glucosidase, Fibrinogen, Phenylalanine 4 Hydroxylase, Alkaline Phosphatase, Glucosylceramidase, Beta Galactosidase, Porphobilinogen Deaminase, Arylsulfatase B, Beta Glucuronidase, Alpha-N-Acetylglucosaminidase, Lysosomal Alpha, Alpha L-Iduronidase, Mannosidase, Phosphatidylcholine Sterol Acyltransferase, N-Sulphoglucosamine Sulphohydrolase, Coagulation Factor X, N-Acetylgalactosamine-6-Sulfatase, Sphingomyelin Phosphodiesterase, iduronate-2-sulfatase, Lysosomal Alpha Glucosidase, Cyclin Dependent Kinase Like 5, Prolow Density Lipoprotein Receptor Related Protein 1, Phenylalanine Ammonia Lyase, Protein Glutamine Gamma Glutamyltransferase K, or Lysosomal Protective Protein encoding gene.

32. The composition of claim 30, wherein the donor molecule comprises a sequence from an acid α-glucosidase, α-L-iduronidase, α-galactosidase, iduronate-2-sulfatase, N-acetylgalactosamine-6-sulfatase, N-acetylgalactosamine-4-sulfatase, a lysophosphatidylcholine metabolism-related protein, preferably phospholipase A2, a T-REC or K-REC related protein, β-glucosidase, β-glucocerebrosidase, arylsulfatase A, Factor VIII, insulin-like growth factor 1 (IGF-1), surfactant protein A, surfactant protein B, aspartyl-β-glucosaminidase, acetyl-CoA α-glucosaminide, acetyl-CoA-arylamine N-acetyltransferase, N-acetylglucosamine-6-sulfatase, N-acetylglucosamine-1-Phosphotransferase, α-N-acetylglucosaminidase, acid ceramidase, aspartacylase, lysosomal acid lipase, acid sphingomyelinase, arylsulfatase B, α-L-fucosidase, galactosylceramidase, galactocerebrosidase, β-galactosidase, protective protein/cathepsin A, β-glucoronidase, heparan N-sulfatase, β-hexosaminidase A, hyaluronidase-1, alpha-D-mannosidase, beta-mannosidase, alpha-neuraminidase, beta-hexosaminidase A, beta-hexosaminidase B, palmitoyl-protein thioesterase, tripeptidyl peptidase I, Battenin, Ceroid-lipofuscinosis neuronal protein 5 (CLN5), Ceroid-lipofuscinosis neuronal protein 6 (CLN6), Ceroid-lipofuscinosis neuronal protein 7 (CLN7), Ceroid-lipofuscinosis neuronal protein 8 (CLN8), (Cathepsin D), cystinosin, cathepsin K, Sialin, Lysosome-associated membrane protein 2 (LAMP2), human growth hormone, follicle-stimulating hormone, erythropoietin, CD-19, a cytokine, a chemokine, IL-10, IGF1, TGF-β, IL-15, CXCR4, IL-4, or a granulocyte colony-stimulating factor (G-CSF) encoding gene.

33. The composition of any one of claims 30-32, wherein the donor molecule comprises a sequence from a gene encoding a protein that is secreted by a cell.

34. The composition of any one of claims 30-33, wherein the donor molecule comprises a sequence that encodes a polypeptide of interest.

35. The composition of any one of claims 30-34, wherein the donor molecule comprises a sequence encoding a 2A self-cleaving peptide.

36. The composition of any one of claims 30-35, wherein the donor molecule comprises a sequence that encodes a signal peptide.

37. The composition of claim 36, wherein the signal peptide is a B2M signal peptide.

38. The composition of any one of claims 30-37, wherein the donor molecule comprises a splice acceptor sequence and a splice donor sequence.

39. The composition of any one of claims 30-38, wherein the donor molecule comprises a splice acceptor sequence, a sequence encoding a polypeptide of interest, a sequence encoding a 2A self-cleaving peptide, a signal peptide, and a splice donor sequence.

40. The composition of any one of claims 30-39, wherein the donor molecule comprises a first homology arm sequence that shares at least 90%, preferably 100%, sequence identity with a first sequence in the B2M gene, and a second homology arm sequence that shares at least 90%, preferably 100%, sequence identity with a second sequence in the B2M gene.

41. The composition of claim 40, wherein the first homology arm sequence and second homology arm sequences are each about 20-50, 50-100, 100-200, 200-500, 500-1000, or 1000-2000 nucleotides in length.

42. The composition of any one of claims 34-41, wherein the polypeptide of interest is a soluble protein.

43. The composition of any one of claims 34-42, wherein the polypeptide of interest is up to 20-50, 50-100, 100-200, 200-500, 500-1000, or 1000-2000 amino acids in length.

44. The composition of any one of claims 28-43, further comprising a tracrRNA molecule.

45. A method for modifying a B2M allele in a cell, the method comprising delivering to the cell the composition of any one of claims 28-44.

46. A method for treating a disorder or disease, the method comprising delivering to a cell of a subject having the disorder or disease the composition of any one of claims 28-44 or delivering to the subject the modified cell of claims 26-27.

47. The method of claim 46, wherein the disorder or disease is Pompe disease, Mucopolysaccharidosis Type I, Fabry disease, Mucopolysaccharidosis type II, Mucopolysaccharidosis type IVA, Mucopolysaccharidosis type VI, Adrenoleukodystrophy, Severe combined immunodeficiency, Gaucher disease, metachromatic leukodystrophy (MLD), primary immune deficiency, hemophilia A, hemophilia B, IGF-1 deficiency, surfactant deficiency, Aspartylglycosaminuria, Sanfilipo syndrome, mucopolysaccharidosis type III, Sanfilippo Syndrome type IIId, I-cell disease, Schindler Disease, Farber disease (FD), Spinal muscular atrophy with progressive myoclonic epilepsy (SMA-PME), Canavan disease, Lysosomal acid lipase deficiency, Niemann-Pick disease, Mucopolysaccharidosis type 6, Fucosidosis, Krabbe disease, GM1 gangliosidosis, mucopolysaccharidosis type IVB (MPS IVB), or galactosialidosis, Sly disease, Mucopolysaccharidosis type III, Late-onset Tay Sachs, Hyaluronidase-1 deficiency, Alpha-mannosidosis, Beta-mannosidosis, Sialidosis, Stanhoff disease, Santavuori-Haltia disease, Jansky-Bielschowsky disease, Batten disease, Neuronal ceroid lipofuscinosis type 5, Neuronal ceroid lipofuscinosis type 6, Neuronal ceroid lipofuscinosis type 7, Neuronal ceroid lipofuscinosis type 8, Congenital cathepsin D deficiency, Cystinosis, Pycnodysostosis, Salla disease, Danon disease, and/or Alpha-1 antitrypsin deficiency.

48. The method of claim 46 or 47, wherein the composition or the modified cell is delivered to a tissue or tumor of the subject.

49. A medicament comprising the composition of any one of claims 28-44 for use in modifying a B2M allele in a cell, wherein the medicament is administered by delivering to the cell the composition of any one of claims 28-44.

50. Use of the composition of any one of claims 28-44 or the modified cell of claims 26-27 for treating ameliorating or preventing a disorder or disease, comprising delivering to a cell of a subject having or at risk of having the disorder or disease the composition of any one of claims 28-44 or delivering to the subject the modified cell of claims 26-27.

51. A medicament comprising the composition of any one of claims 28-44 or the modified cell of claims 26-27 for use in treating ameliorating or preventing a disorder or disease, wherein the medicament is administered by delivering to a cell of a subject having or at risk of having the disorder or disease the composition of any one of claims 28-44 or delivering to the subject the modified cell of claims 26-27.

52. The method of claims 46-48, the use of claim 50, or the medicament of claim 51, wherein the disorder or disease is a lysosomal storage disease.

53. The method of claims 46-48, the use of claim 50, or the medicament of claim 51, wherein the disorder or disease is Pompe disease, Mucopolysaccharidosis Type I, Fabry disease, Mucopolysaccharidosis type II, Mucopolysaccharidosis type IVA, Mucopolysaccharidosis type VI, Adrenoleukodystrophy, Severe combined immunodeficiency, Gaucher disease, metachromatic leukodystrophy (MLD), primary immune deficiency, hemophilia A, hemophilia B, IGF-1 deficiency, surfactant deficiency, Aspartylglycosaminuria, Sanfilipo syndrome, mucopolysaccharidosis type III, Sanfilippo Syndrome type IIId, I-cell disease, Schindler Disease, Farber disease (FD), Spinal muscular atrophy with progressive myoclonic epilepsy (SMA-PME), Canavan disease, Lysosomal acid lipase deficiency, Niemann-Pick disease, Mucopolysaccharidosis type 6, Fucosidosis, Krabbe disease, GM1 gangliosidosis, mucopolysaccharidosis type IVB (MPS IVB), or galactosialidosis, Sly disease, Mucopolysaccharidosis type III, Late-onset Tay Sachs, Hyaluronidase-1 deficiency, Alpha-mannosidosis, Beta-mannosidosis, Sialidosis, Stanhoff disease, Santavuori-Haltia disease, Jansky-Bielschowsky disease, Batten disease, Neuronal ceroid lipofuscinosis type 5, Neuronal ceroid lipofuscinosis type 6, Neuronal ceroid lipofuscinosis type 7, Neuronal ceroid lipofuscinosis type 8, Congenital cathepsin D deficiency, Cystinosis, Pycnodysostosis, Salla disease, Danon disease, and/or Alpha-1 antitrypsin deficiency.

54. The method of claims 46-48, the use of claim 50, or the medicament of claim 51, wherein the disorder or disease is a disorder or disease of the blood, lungs, brain, liver, guts, intestines, bones, muscles, or central nervous system, or is an inflammatory disease or autoinflammatory disease.

55. The medicament of claim 51, wherein the medicament is an enzyme replacement therapy.

56. A method of treating a disease or disorder in a subject, wherein the method is an immunotherapy comprising delivering to the subject the modified cell of claims 26-27.

57. The method of claim 56, wherein the disease or disorder is cancer.

58. The composition of any one of claims 28-44 or the modified cell of claims 26-27 for use in treating ameliorating or preventing a disorder or disease.

59. A composition, method, process, kit, or use as characterized by one or more elements disclosed herein.
